(19) United States
(12) Patent Application Publication
(10) Pub. No.: US 2025/0127941 A1
(43) Pub. Date: Apr. 24, 2025

(54) DETECTION AND LOCALIZATION OF INTERNAL BLEEDING

(71) Applicants: VANDERBILT UNIVERSITY, Nashville, TN (US); CUMBERLAND EMERGING TECHNOLOGIES, INC., Nashville, TN (US)

(72) Inventors: Seth Karp, Nashville, TN (US); Leo PAVLIV, Cary, NC (US); Jerri Rook, Nashville, TN (US); Andrew VILA, Nashville, TN (US)

(21) Appl. No.: 18/728,193

(22) PCT Filed: Jan. 11, 2023
(86) PCT No.: PCT/IB2023/050269

§ 371 Date: Jul. 11, 2024

Related U.S. Application Data
(60) Provisional application No. 63/298,911, filed on Jan. 12, 2022.

(51) Int. Cl. A61K 51/08; C07B 59/00

(52) U.S. Cl. CPC A61K 51/088; C07B 59/008; A61K 2123/00; C07B 2200/05

(57) ABSTRACT

Systems, methods, and kits useful for detecting and locating internal bleeding, such as gastrointestinal bleeding, during active or inactive bleeding. In some embodiments, the systems, methods, and kits make use of a single photon emission computed tomography (SPECT) tracer. The kit for preparing a technetium-99m (99mTc)-labeled composition for targeting an injured site in a blood vessel, comprising: -a conjugated human fibrinogen, wherein the human fibrinogen is bonded to a chelating agent selected from the group consisting of diethylene triamine pentaacetate (DTPA) dianhydride.

### CROSS-REFERENCE TO RELATED APPLICATIONS

This application claims priority to and the benefit of U.S. Provisional Application No. 63/298,911, filed Jan. 12, 2022, the entire contents of which is hereby incorporated by reference.

### FEDERALLY SPONSORED RESEARCH OR DEVELOPMENT

This invention was made with government support under Contract No. 1R41DK106779-01A1 awarded by the National Institute of Diabetes and Digestive and Kidney Diseases. The government has certain rights in the invention.

### TECHNICAL FIELD

The presently-disclosed subject matter generally relates to medical diagnostic tools for use in imaging internal bleeding in a subject. In particular, certain embodiments of the presently-disclosed subject matter relate to systems, methods, and kits useful for detecting and locating gastrointestinal bleeds, even during episodes of minimal bleeding. In some embodiments, the systems, methods, and kits make use of an effective single photon emission computed tomography (SPECT) tracer.

### BACKGROUND

Gastrointestinal (GI) bleeding is a significant medical problem in the United States. Approximately 500,000 people each year in the United States are hospitalized with GI bleeds, including both upper and lower GI, resulting in tens of billions of dollars of healthcare spending annually [1]. Although the mortality rate for GI bleeding has significantly decreased over the past two decades due to advances in medical and endoscopic therapies, studies still report mortality rates up to 10% for both upper and lower GI bleed (i.e., 50,000 patients per year) [2, 3].

Bleeding in the lower gastrointestinal tract is surprisingly difficult to locate. Indeed, about 50% of the time the tests that are now used to diagnose and locate gastrointestinal bleeding will fail. Such failures of existing technology occur because it is common for bleeding from a single site to stop and restart multiple times [2], and available tests require a minimum rate of bleeding at the time of testing.

Based on an initial clinical evaluation, if a lower GI bleed is suspected, current American College of Gastroenterology guidelines [4] recommend a diagnostic colonoscopy to identify the bleeding site. Likewise, if an upper GI bleed is suspected, a diagnostic endoscopic procedure is prescribed [5]. However, colonoscopies and endoscopies are often inconclusive because, as noted above, GI bleeds are notoriously unstable—vacillating between active bleeding and temporary hemostasis [2]. This feature of GI bleeds allows them to evade visual inspection.

Even active bleeds can often go undetected by visual methods due to other confounding issues including inadequate bowel preparation and the timing of imaging (where adequate bowel preparation can take more than 24 hours) [6].

GI bleeds are classified as overt, obscure, or occult. Overt GI bleeding is visible (e.g., bloody emesis, hematochezia, and melena), whereas obscure GI bleeding refers to recurrent bleeding in which the source cannot be identified by upper endoscopy, colonoscopy, or small bowel radiography. Obscure bleeding can be either overt or occult, where occult bleeding is not visible to the patient or physician.

The current standard of care as defined by the American College of Gastroenterology involves upper endoscopy or colonoscopy as a first-line diagnostic for UGIB and LGIB, respectively. When endoscopic or colonoscopic inspection is either inconclusive or not feasible, second-line diagnostic techniques are available. These include contrast angiography and/or agents like technetium-tagged red blood cells (i.e., red blood cell scintigraphy), or other diagnostic tracers used in combination with an imaging modality as described in the competition section below. However, all of these techniques require that the bleed be active at the time of inspection to enable accurate detection. Furthermore, the tagged red blood cell scan, which also relies on single-photon emission computed tomography (SPECT) imaging for readout, was once a commonly used technique but has fallen out of favor and is no longer recommended by current guidelines due to its inability to detect an inactive bleed.

The lack of an accurate, non-invasive, and rapid method for locating a GI bleeding site, results in up to 50,000 otherwise avoidable deaths occurring each year. Additionally, the lack of tools to provide a rapid, definitive diagnosis leads to unnecessary additional testing, prolonged hospitalization, and increased risk for rebleeding.

Once a bleeding site is located, therapeutic intervention (e.g., endoscopic hemostasis) is usually straightforward [7]. However, it is the accurate and timely localization of the bleeding site that remains a major unmet medical need for effectively treating and reducing GI-bleeding associated mortality.

Accordingly, there remains a need in the art for technology that can accurately and rapidly diagnose and locate both active and inactive bleeds, such as those occurring in the gastrointestinal system.

### SUMMARY OF THE INVENTION

Disclosed herein are kits for preparing a technetium-99m (^{99m}Tc)-labeled composition for targeting an injured site in a blood vessel. For instance, the kit may comprise a human fibrinogen; a chelating agent selected from the group consisting of diethylene triamine pentaacetate (DTPA) dianhydride, p-SCN-Bz-DTPA, HYNIC, and a combination thereof, and a reducing agent. The kit may also comprise a conjugated human fibrinogen, wherein the human fibrinogen is bonded to a chelating agent selected from the group consisting of diethylene triamine pentaacetate (DTPA) dianhydride, p-SCN-Bz-DTPA, HYNIC, and a combination thereof, and a reducing agent.

Also disclosed are methods for preparing a technetium-99m (^{99m}Tc)-labeled fibrinogen composition for targeting an injured site in a blood vessel, comprising preparing or providing a conjugated human fibrinogen and a reducing agent using a kit disclosed herein; combining the conjugated human fibrinogen with a ^{99m}Tc-containing solution, wherein the reducing agent is combined with the conjugated human fibrinogen before, after, or at the same time as the ^{99m}Tc-containing solution; incubating the combined conjugated human fibrinogen, reducing agent, and ^{99m}Tc-containing solution to give the ^{99m}Tc-labeled fibrinogen composition; and optionally checking the radiochemical purity of the ^{99m}Tc-labeled fibrinogen composition. Compositions prepared according to these methods are also disclosed.

Also disclosed are methods for detecting an internal bleeding site in a subject, comprising administering to the subject the radiopharmaceutical compositions disclosed herein and imaging a region of interest of the subject, thereby detecting the internal bleeding site in the subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A includes HPLC chromatograms for a first batch (201022) of prepared 99mTc-DTPA-DA-Fibrinogen after 30 min equilibrated column, injection 20 uL (108uCi), where the top chromatography is UV absorbance (mAU), and the bottom chromatography is radioactivity (mV).

FIG. 1B includes HPLC chromatograms for a first batch (201023) of prepared 99mTc-DTPA-DA-Fibrinogen after 30 min equilibrated column, injection 50 uL (210uCi), where the top chromatography is UV absorbance (mAU), and the bottom chromatography is radioactivity (mV).

FIG. 1C includes HPLC chromatograms for a first batch (201027) of prepared 99mTc-DTPA-DA-Fibrinogen after 30 min equilibrated column, injection 40 uL (323uCi), where the top chromatography is UV absorbance (mAU), and the bottom chromatography is radioactivity (mV).

FIG. 2A includes representative SPECT/CT images acquired 30 minutes post-injection after Rat #1 was injected with ^{99m}Tc-Fibrinogen.

FIG. 2B includes representative SPECT/CT images acquired 1 hr post-injection after Rat #2 was injected with ^{99m}Tc-Fibrinogen.

FIG. 2C includes representative SPECT/CT images of a rat acquired 2 hours after injection with ^{99m}Tc—O₃ as a control compound.

FIG. 3A includes a representative axial SPECT/CT image of a rat that was acquired following injection of ^{99m}Tc-Fibrinogen.

FIG. 3B includes a representative coronal SPECT/CT image of a rat that was acquired following injection of ^{99m}Tc-Fibrinogen.

FIG. 3C includes a representative sagittal SPECT/CT image of a rat that was acquired following injection of ^{99m}Tc-Fibrinogen.

FIG. 3D includes a representative axial SPECT/CT image of a rat that was acquired following injection of ^{99m}TcO₄ as a control compound.

FIG. 3E includes a representative coronal SPECT/CT image of a rat that was acquired following injection of ^{99m}TcO₄ as a control compound.

FIG. 3F includes a representative sagittal SPECT/CT image of a rat that was acquired following injection of ^{99m}TcO₄ as a control compound.

FIG. 4A includes an axial SPECT/CT image that was acquired after injection with ^{99m}Tc-Fibrinogen in a rat with gastric injury.

FIG. 4B includes a coronal SPECT/CT image that was acquired after injection with ^{99m}Tc-Fibrinogen in a rat with gastric injury.

FIG. 4C includes a sagittal SPECT/CT image that was acquired after injection with ^{99m}Tc-Fibrinogen in a rat with gastric injury.

FIG. 4D includes an axial SPECT/CT image that was acquired after injection with ^{99m}Tc-Fibrinogen in a normal rat.

FIG. 4E includes a coronal SPECT/CT image that was acquired after injection with ^{99m}Tc-Fibrinogen in a normal rat.

FIG. 4F includes a sagittal SPECT/CT image that was acquired after injection with ^{99m}Tc-Fibrinogen in a normal rat.

FIG. 4G includes an axial SPECT/CT image that was acquired after injection with ^{99m}Tc-Fibrinogen in a rat that had a sham surgery.

FIG. 4H includes a coronal SPECT/CT image that was acquired after injection with ^{99m}Tc-Fibrinogen in a rat that had a sham surgery.

FIG. 4I includes a sagittal SPECT/CT image that was acquired after injection with ^{99m}Tc-Fibrinogen in a rat that had a sham surgery.

FIG. 5 includes results from ex vivo SPECT/CT imaging that was performed on organs taken from rats with gastric injury or sham injury as described in Table 11. The labeling indicates the rat number followed by I for Injury, C for Control, L for Liver and K for Kidney.

FIG. 6 includes results from ex vivo analysis of organs taken from normal healthy rats that were injected with ^{99m}Tc-Fibrinogen immediately upon arrival (black bars) or 3 hours after arrival (hatched bars). Rats were euthanized 1 hour post-injection of radiotracer. *Counts obtained from the kidneys were above the range for the gamma counter.

FIG. 7A shows a representative SPECT/CT image of a rat injected with 250 μCi ^{99m}Tc-DTPA-DA-Fibrinogen in a gastric mucosal stomach injury model.

FIG. 7B shows a representative SPECT/CT image of a rat injected with 250 μCi ^{99m}Tc-DTPA-DA-Fibrinogen in a colon injury model.

FIG. 7C shows a representative SPECT/CT image of a rat injected with 250 μCi ^{99m}Tc-DTPA-DA-Fibrinogen in a sham surgery.

FIG. 8 shows Standard Uptake Values (SUV) from SPECT/CT images of rats injected with 250 uCi ^{99m}Tc-DTPA-DA-Fibrinogen with 5 minute pretreatment time.

FIG. 9A shows a representative SPECT/CT image of a rat injected with 250 μCi ^{99m}Tc-DTPA-DA-Fibrinogen in a sham surgery.

FIG. 9B shows a representative SPECT/CT image of a rat injected with 250 μCi ^{99m}Tc-DTPA-DA-Fibrinogen in a sham surgery.

FIG. 9C shows a representative SPECT/CT image of a rat injected with 250 μCi ^{99m}Tc-DTPA-DA-Fibrinogen in a colon injury model.

FIG. 9D shows a representative SPECT/CT image of a rat injected with 250 μCi ^{99m}Tc-DTPA-DA-Fibrinogen in a colon injury model.

FIG. 10 shows Standard Uptake Values (SUV) from SPECT/CT images of rats injected with 250 μCi ^{99m}Tc-DTPA-DA-Fibrinogen with 5 minute pretreatment time.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes systems, methods, and kits useful for accurately and rapidly diagnosing and locating both active and inactive internal bleeding, such as gastrointestinal (GI) bleeds.

Some embodiments of the presently-disclosed subject matter include a kit for diagnosing and locating both active and inactive internal bleeding, such as gastrointestinal (GI) bleeding. In some embodiments, the kit can include fibrinogen that has been modified to facilitate stable and efficient labeling with technetium-99m (^{99m}TC). The kit can be used by one of ordinary skill in the medical arts, for example, by a radiopharmacy upon the order of a physician treating a subject suspected of having internal bleeding, such as a GI bleed.

In some embodiments of the presently-disclosed subject matter, a method and/or kit is provided for use in synthesizing a fibrinogen radiopharmaceutical composition, which includes Technetium-99m (^{99m}Tc), fibrinogen, and a chelating agent, such as diethylene triamine pentaacetic acid (DTPA) dianhydride, p-SCN-Bz-DTPA, or HYNIC, linking the ^{99m}Tc and the fibrinogen. For example, a method and/or kit is provided for use in the synthesis of a fibrinogen radiopharmaceutical composition that includes fibrinogen, such as GMP-produced human fibrinogen, which is covalently modified with a chelating agent, such as DTPA dianhydride, p-SCN-Bz-DTPA, or HYNIC. Conjugated fibrinogen can be modified to add 99m-technetium using stannous chloride (SnCl₂).

^{99m}Tc is a metastable nuclear isomer of technetium-99, which can be used as a radioactive tracer and can be detected in the body by currently-available medical equipment (e.g., gamma cameras). ^{99m}Tc has a relatively short half-life, and will remain in a subject (e.g., human subject) for only about 24 hours, allowing for medical scanning processes and data collection, while dispersing rapidly to minimize the subject's radiation exposure.

Fibrinogen is a glycoprotein complex. During tissue and vascular injury, it is converted to fibrin and then to a fibrin-based blood clot. Fibrin clots function primarily to occlude blood vessels to stop bleeding. In the context of the presently-disclosed subject matter, fibrinogen can serve as a targeting agent, directing the composition to the site of an injured blood vessel. As will be recognized by the skilled artisan, chelating agents are chemical compounds that react with metal ions to form a stable complex. For example, DTPA dianhydride, p-SCN-Bz-DTPA, and HYNIC are high affinity chelating agents for ^{99m}Tc, and can be used to link the fibrinogen to ^{99m}Tc, to create a composition having a targeting component and a radiotracer component.

Once synthesized, the composition is ready for administration. For example, the composition can be injected into the subject. Once injected into a subject, the fibrinogen component targets the composition to the injury site, where it accumulates.

The subject can then undergo imaging, such as single-photon emission computed tomography (SPECT) imaging. The ^{99m}Tc fibrinogen radiopharmaceutical composition allows the accumulated composition to be images, thereby revealing the site of active or inactive bleeding, such as GI bleeding.

In some embodiments of the presently-disclosed subject matter systems, methods, and kits are provided for preparing a fibrinogen radiopharmaceutical composition including fibrinogen, chelating agent, and technetium-99m (99mTc-conjugated-Fibrinogen), which accumulates at an injured blood vessel wall as part of an adherent clot, and the composition can be used to locate bleeding sites even after bleeding has stopped. The composition can be prepared, for example, using an U.S. Food and Drug Administration (FDA)-approved source of fibrinogen. The composition can be used, for example, with established imaging technologies, such as SPECT imaging.

### Fibrinogen Radiopharmaceutical Compositions

Disclosed is a fibrinogen radiopharmaceutical composition containing (i) radiolabeled fibrinogen in a biocompatible carrier; (ii) a reducing agent; (iii) a buffering agent; and (iv) a chelating agent.

The term “radiopharmaceutical” is a term well known to the person skilled in the art of nuclear medicine. The majority of radiopharmaceuticals are used for in vivo imaging, and comprise a radionuclide having emissions suitable for detection, typically by single-photon emission computed tomography (SPECT) or positron emission tomography (PET). Such a radionuclide together with a biocompatible carrier, in a form suitable for mammalian administration is a “radiopharmaceutical composition”. See the “Handbook of Radiopharmaceuticals” (Welch & Redvanly, Eds. Wiley 2003) for an overview of radiopharmaceuticals.

As used herein, the term “fibrinogen” means a protein that is converted into fibrin by the action of thrombin, especially during blood clot formation. The fibrinogen can be a natural substance produced by a human. Fibrinogen isolated from an animal, including but not limited to, a rat, mouse, pig, sheep, goat, horse, dog or monkey, is also encompassed. The fibrinogen can also be a recombinant product produced by a recombinant host, such as a recombinant bacterium, from a fibrinogen coding sequence of an animal, including but not limited to, human, rat, mouse, pig, sheep, goat, horse, dog or monkey. For example, human fibrinogen may be isolated from body fluids, in particular the milk, of transgenic animals. See, e.g., U.S. Pat. No. 5,639,940. The fibrinogen further includes any structural and/or functional derivative of the naturally occurring fibrinogen, such as a fragment of or a chemically modified fibrinogen, that maintains the biological activity of the fibrinogen.

Fibrinogen useful in the disclosed systems, methods, and kits may be produced by methods known in the art, for instance, as described in U.S. Pat. Nos. 9,371,355, 9,938,318, 10,112,972, and 11,401,300, all of which are hereby incorporated by reference. In one embodiment, the fibrinogen is human fibrinogen. Several plasma-derived human fibrinogen concentrates are available and may be useful in the present disclosure, such as those sold under the trademark FIBRYGA® (Octapharma AG, Lachen, Switzerland), RIASTAP®/HAEMOCOMPLETTAN® P (CSL Behring GmbH, Marburg, Germany), FIBCLOT®/CLOTTAFACT® (LFB, Les Ulis, France), FIBRINOGEN HT (Benesis, Osaka, Japan), FIBRORAAS (Shanghai RAAS, Shanghai, China), and GCC-FIBRINOGEN. See, e.g., [23-24]. For instance, the human fibrinogen may be a lyophilized powder for reconstitution for intravenous use sold under the trademark FIBRYGA®, where the nominal composition is 20 mg/mL human fibrinogen, 6 mg/mL sodium chloride, 1.5 mg/mL sodium citrate dihydrate, 10 mg/mL glycine, and 10 mg/mL L-arginine hydrochloride. The human fibrinogen may comply with the U.S. Food and Drug Administration's Good Manufacturing Practice (GMP) guidelines. See 21 C.F.R. §§ 210-211.

The fibrinogen useful in the disclosed systems, methods, and kits may contain additional components, such as impurities or excipients. The fibrinogen may further comprise, for example, sodium chloride, sodium citrate dihydrate, sodium citrate, sodium hydroxide, hydrochloric acid, glycine, isoleucine, lysine hydrochloride, L-arginine hydrochloride, fibronectin, Von Willebrand Factor (VWF), vitronectin, albumin, factor XIII, D-dimer, fibrinopeptide A, plasminogen, or a combination thereof. In some embodiments, there may be no detectable vitronectin in the human fibrinogen. The concentration of fibrinopeptide A in the fibrinogen may be below the limit of human plasma (less than about 7.6 ng/mL). In another embodiment, the fibronectin concentration in the fibrinogen may be below the concentration in human plasma (about 300 μg/mL). In another embodiment, the VWF concentration in the fibrinogen may be equal to or below the concentration in human plasma (about 0.36 to about 1.57 U/mL).

The fibrinogen may be substantially free of albumin. The phrase “substantially free,” as used herein, refers to fibrinogen that has significantly reduced levels of albumin, for instance, albumin is not added to the fibrinogen but may be present otherwise, such as an impurity or contaminant. In some embodiments, the fibrinogen may contain less than 50% (w/w), from 0.01 to 40% (w/w), from 0.01 to 30% (w/w), from 0.01 to 20% (w/w), from 0.01 to 10% (w/w), from 0.01 to 5.0% (w/w), from 0.50 to 3.0% (w/w), or about 1.9% albumin. In some embodiments, the fibrinogen may contain from 0.001 to 9.00 mg/mL, from 0.001 to 5.00 mg/mL, from 0.001 to 1.00 mg/mL, from 0.100 to 1.00 mg/mL, or about 0.42 mg/mL albumin.

The fibrinogen may contain factor XIII, a protein complex circulating in plasma in an activity of 0.77-1.69 U/mL (mean value 1.2±0.3 U/mL). Factor XIII activity in the fibrinogen may be equal to or up to 2, 3, 4, or 5 times higher than the activity in human plasma. In one embodiment, the activity of factor XIII in the fibrinogen is from 2 to 4 times higher than the activity in human plasma.

The fibrinogen may be used in the fibrinogen radiopharmaceutical composition at a concentration of from 5.0 to 15 mg/mL. For instance, the concentration may be 5.0, 6.0, 7.0, 8.0, 9.0, 10, 11, 12, 13, 14, or 15 mg/mL. In one embodiment the fibrinogen is present in the fibrinogen radiopharmaceutical composition at a concentration of 10 mg/mL.

The fibrinogen may be labeled with a radionuclide such as, for example, ⁵⁵Co, ⁶⁴Cu, or ^{99m}Tc. In one embodiment, the fibrinogen is labelled with ^{99m}Tc. Accordingly, in one embodiment, the fibrinogen radiopharmaceutical composition comprises (i)^{99m}Tc-labeled fibrinogen in a biocompatible carrier; (ii) a reducing agent; (iii) a buffering agent; and (iv) a chelating agent.

A “biocompatible carrier” is a fluid, especially a liquid, in which the radiopharmaceutical is suspended or dissolved, such that the composition is physiologically tolerable, i.e. can be administered without toxicity or undue discomfort. The biocompatible carrier is suitably an injectable carrier liquid such as sterile, pyrogen-free water for injection. The biocompatible carrier may also be an aqueous solution of one or more tonicity agents, such as salts of plasma cations with biocompatible counterions (e.g. sodium chloride, potassium chloride), sugars (e.g. glucose or sucrose), sugar alcohols (e.g. sorbitol, mannitol, inositol), glycols (e.g. glycerol), or other non-ionic polyol materials (e.g. polyethylene glycols, propylene glycols and the like). The biocompatible carrier may also comprise biocompatible organic solvents such as ethanol. Such organic solvents are useful to solubilize more lipophilic compounds or formulations.

In one embodiment, the biocompatible carrier is pyrogen-free water for injection or isotonic saline. In another embodiment, the biocompatible carrier comprises an aqueous solvent. In yet another embodiment, the biocompatible carrier comprises isotonic saline solution. For instance, sodium chloride may be used in the fibrinogen radiopharmaceutical composition at a concentration of from 0.02 to 0.2 M or from 0.03 to 0.07 M.

As used herein, “reducing agent” is a compound that reacts with a radionuclide, which is typically obtained as a relatively unreactive, high oxidation state compound, to lower its oxidation state by transferring electron(s) to the radionuclide, thereby making it more reactive, which is non-toxic at the required dosage and hence suitable for administration to the mammalian body, especially the human body. Reducing agents useful in the fibrinogen radiopharmaceutical compositions include, but are not limited to, stannous chloride, stannous tartrate, stannous fluoride, stannous phosphate, formamidine sulfinic acid, sodium dithionite, sodium bisulphite, ascorbic acid, cysteine, phosphines, and cuprous salts. In one embodiment, the reducing agent is a stannous salt, such as stannous chloride or stannous tartrate.

The reducing agent may be present in the composition at a concentration of from 1 to 10 mM, from 2 to 8 mM, from 3 to 6 mM, or from 4 to 5 mM. The reducing agent, such as stannous chloride, may be present at, for instance, 4.0, 4.1, 4.2, 4.3, 4.4, 4.5, 4.6, 4.7, 4.8, 4.9, or 5 mM.

The disclosed fibrinogen radiopharmaceutical compositions may comprise at least one chelating agent, such as ethylenediaminetetraacetic acid (EDTA), Ethyleneglycol Bis(2-Aminoethyl Ether)-N,N,N′,N′ Tetraacetic Acid (EGTA), diethylenetriaminepentaacetic acid (DTPA), DTPA dianhydride, triethylenetetraaminehexaacetic acid (TTHA), trans-1,2-diaminocyclohexane-N,N,N′,N′-tetraacetic acid (CDTA), ethylenediaminedisuccinic acid (EDDS), diethylenetriaminepenta(methylene phosphonic acid) (DTPMP), N-hydroxyethylethylenediaminetri-acetic acid (HEDTA), N-hydroxyethyliminodiacetic acid (HEIDA), dihydroxyethylglycine (DHEG), ethylenediaminetetrapropionic acid (EDTP), pentasodium pentetate, pentetic acid, dihydroxyethyl glycine, citric acid, succinic acid, tartaric acid, and analogs thereof including derivatives of DTPA including cyclohexane-1,2-diamine-N,N,N′,N′-tetraacetate (CHX-DTPA), p-isothiocyanatobenzyl diethylenetriaminepentaacetic acid (p-SCN-Bz-DTPA), Diethylenetriamine-N,N,N″,N″-tetra-tert-butyl acetate-N′-acetic acid (Tetra-t-Bu-DTPA), activated esters of DTPA, and other bifunctional chelators including 1,4-methyl-benzyl isothiocyanate diethylenetriamine pentaacetic acid (111In-MX-DTPA), 1,4,8,11-tetraazacyclotetradecane-1,4,8,11-tetraacetic acid (TETA), 1,4,7,10-Tetraazacyclododecane-N,N′,N″,N″′-tetraacetic acid (DOTA), and analogs of DOTA, such as DOTA-NHS-ester, other activated esters of DOTA, 1,4,7-Triazacyclononane-1,4,7-triacetic acid (NOTA). Other useful chelators may include 6-hydrazinonicotinamide (HYNIC), sodium gluconate, pendetide, and mercaptoacetyltriglycine (MAG3). The chelating agent may be used in its acid form, but it may also be used as one of its salts, for instance calcium and/or zinc trisodium salts of DTPA, edetate calcium disodium, edetate disodium, edetate sodium, edetate trisodium, edetate dipotassium, sodium ascorbate, and sodium 5-sulfosalicyclate. In one embodiment, the radiopharmaceutical composition contains DTPA dianhydride. In another embodiment, the radiopharmaceutical composition contains p-SCN-Bz-DTPA. In yet another embodiment, the radiopharmaceutical composition contains HYNIC. The chelating agent, such as DTPA dianhydride, p-SCN-Bz-DTPA, or HYNIC, may be present at a concentration of from 1 to 5 μm or at a concentration of 3.5 μm. In some embodiments, the molar ratio of chelating agent, such as DTPA dianhydride, p-SCN-Bz-DTPA, or HYNIC, to fibrinogen is from 1:1 to 30:1. For instance, the molar ratio of chelating agent, such as DTPA dianhydride, p-SCN-Bz-DTPA, or HYNIC, to fibrinogen may be about 1:1, 5:1, 10:1, 12:1, or 30:1. The “fibrinogen” and the “chelating agent” of the radiopharmaceutical composition may be separate, a chelate bond may exist between the fibrinogen and the chelating agent, or both. The separate use of these terms throughout the disclosure is not meant to exclude a chelate bond existing between the fibrinogen and chelating agent in the radiopharmaceutical composition.

The disclosed fibrinogen radiopharmaceutical compositions may optionally also comprise at least one antioxidant. “Antioxidant” refers to any compound which protects an active ingredient from reaction with oxygen. Antioxidants include, but are not limited to, ascorbic acid, 5-sulfosalicyclic acid, gentisic acid, nitriloacetate (NTA), sulfites, methionine, NAC, glutathione, lipoic acid, butylated hydroxytoluene (BHT), and cysteine. In one embodiment, the radiopharmaceutical composition contains ascorbic acid. The antioxidant, such as ascorbic acid, may be present at a concentration of from 5 to 40 mM or at a concentration of 20 mM.

The fibrinogen radiopharmaceutical composition may also contain a buffering agent to ensure that the optimum pH is maintained for (i)^{99m}Tc radiolabeling of fibrinogen, (ii) post-reconstitution stability, and/or (iii) suitability for patient administration. Radiopharmaceutical compositions are preferably formulated such that the pH of the solution in water or saline is about a physiological pH, such as a pH of from 7.0 to 8.6 or from 7.4 to 8.0. In one embodiment, the pH of the radiolabeled fibrinogen composition is 7.4, 7.5, 7.6, 7.7, 7.8, 7.9, or 8.0.

Suitable buffering agents include, but are not limited to, pharmaceutically acceptable buffers, such as phosphate buffers, borate buffers, citrate buffers, tricine, TRIS, acetate buffers, hydrochloric acid, and pharmaceutically acceptable bases, such as sodium carbonate, sodium bicarbonate, arginine, histidine, cysteine, glycine, diethanolamine, or mixtures thereof. In one embodiment, the fibrinogen radiopharmaceutical composition comprises phosphate-buffered saline (PBS) at a concentration of from 0.025 to 0.2 M, pH 7.0 to 8.4. In another embodiment, the PBS is present at a concentration of from 0.08 to 0.12 M, pH 7.4 to 8.0.

The radiopharmaceutical composition may optionally further comprise at least one pharmaceutical excipient such as a lyophilization aid, stabilization aid, solubilizing aid, bacteriostat, or a combination thereof. Lyophilization aids useful for the preparation of the radiopharmaceutical composition include but are not limited to inorganic salts such as sodium chloride and water soluble sugars or sugar alcohols such as mannitol, maltose, sucrose, lactose, trehalose, sorbitol, dextran, Ficoll, and polyvinylpyrrolidine (PVP). Stabilization aids useful for the preparation of the radiopharmaceutical composition include but are not limited to ascorbic acid, cysteine, monothioglycerol, sodium bisulfite, sodium metabisulfite, gentisic acid and inositol. Solubilization aids useful for the preparation of the radiopharmaceutical composition include but are not limited to a cyclodextrin (e.g., hydroxypropyl-β-cyclodextrin or sulfobutyl ether β-cyclodextrin), ethanol, glycerin, polyethylene glycol, propylene glycol, polyoxyethylene sorbitan monooleate, sorbitan monooleate, polysorbates, poly(oxyethylene)poly(oxypropylene)poly(oxy-ethylene) block copolymers (Pluronics) and lecithin. Bacteriostats useful for the preparation of the radiopharmaceutical composition include but are not limited to benzyl alcohol, benzalkonium chloride, chlorobutanol, phenyl alcohol, and methyl, propyl or butyl paraben.

When the radiopharmaceutical composition is administered to a subject, a suitable amount of radioactivity to be used is in the range from 5 to 50 mCi per 70 kg body weight, or from 10 to 20 mCi per 70 kg body weight.

Exemplary fibrinogen radiopharmaceutical compositions may be prepared using the following components:

| Fibrinogen | 1-15 mg/mL, such as 10 mg/mL |
|---|---|
| DTPA dianhydride | 1-1,500 nmol/mL, such as 350 nmol/mL |
| Stannous Chloride | 0.0005-10 μmol/mL, such as 4.7 μmol/mL |
| Phosphate-Buffered Saline | 0.08-0.12 mmol/mL, pH 7.4-8.0 |
| Tc-99m | 5 to 50 mCi, such as 10 to 20 mCi |
| Optionally, Sodium Chloride | 0.03-0.07 mmol/mL |
| Optionally, Ascorbic Acid | 5-40 μmol/mL, such as 20 μmol/mL |
| pH | 7.4-8.0 |

| human fibrinogen | 1-15 mg/mL, such as 10 mg/mL |
|---|---|
| (FIBRYGA ®) | |
| p-SCN-Bz-DTPA | 1-1,500 nmol/mL, such as 350 nmol/mL |
| Stannous Chloride | 0.0005-10 μmol/mL, such as 4.7 μmol/mL |
| Phosphate-Buffered Saline | 0.08-0.12 mmol/mL, pH 7.4-8.0 |
| Tc-99m | 5 to 50 mCi, such as 10 to 20 mCi |
| Optionally, Sodium Chloride | 0.03-0.07 mmol/mL |
| Optionally, Ascorbic Acid | 5-40 μmol/mL, such as 20 μmol/mL |
| pH | 7.4-8.0 |

### Diagnostic Kits and Administration

Also disclosed are non-radioactive kits for the preparation of the fibrinogen radiopharmaceutical compositions, said kit comprising at least one suitable container having a formulation contained therein, said formulation comprising: (i) fibrinogen, (ii) a reducing agent, (iii) a buffering agent; and (iv) a chelating agent. In relation to the kit, the terms “fibrinogen,” “reducing agent,” “buffering agent,” “chelating agent” and examples thereof are as defined above for the radiopharmaceutical composition. Also, as described above for the radiopharmaceutical composition, the “fibrinogen” and the “chelating agent” of the kits may be separate, a chelate bond may exist between the fibrinogen and the chelating agent, or both. The separate use of these terms throughout the disclosure is not meant to exclude a chelate bond existing between the fibrinogen and chelating agent in the kit.

A “suitable container” for use in the kit is one which does not interact with any components of the radiopharmaceutical formulation, permits maintenance of sterile integrity, plus allows for an inert headspace gas (e.g. nitrogen or argon), while also permitting addition and withdrawal of solutions by syringe. Such containers may be liquid-tight ampoules and vials, the seal being provided by a liquid-tight or gas-tight closure such as a lid, stopper, or septum. In one embodiment, such container is a septum-sealed vial, wherein the gas-tight closure is crimped on with an overseal (typically of aluminum). Such containers have the additional advantage that the closure can withstand vacuum if desired, for example to change the headspace gas or degas solutions and can withstand an overpressure, for example to aid in the removal of the solution from the container. The gas-tight seal is suitable for puncturing with a hypodermic needle. In one embodiment, such container is a pharmaceutical grade vial. The vial may be suitably made of a pharmaceutical grade material, such as glass or plastic. The glass of the container may optionally be coated to suppress leachables from the glass, such as with silica (SiO₂).

In one embodiment, a container of the kit is provided with a closure suitable for puncturing with a hypodermic needle whilst maintaining seal integrity. The closure may be coated on those of its surface(s) which are in contact with the container contents, such as with ethylene-tetrafluoroethylene copolymer (ETFE) or modified versions thereof. The closure body as distinct from the coating thereon, may be made of a synthetic, elastomeric polymer. The closure body may be made of, for instance, chlorinated or brominated butyl rubber, or neoprene, since such polymers have low oxygen permeability. In one embodiment, the closure body is made of chlorinated butyl rubber.

The non-radioactive kit may optionally further comprise additional components such as at least one pharmaceutical excipient such as a lyophilization aid, stabilization aid, solubilizing aid, bacteriostat, or a combination thereof, as defined above. The inclusion of one or more optional components in the formulation will frequently improve the ease of synthesis of the radiopharmaceutical by the practicing end user, the ease of manufacturing the kit, the shelf-life of the kit, or the stability and shelf-life of the radiopharmaceutical.

The one or more suitable containers that contain all or part of the formulation can independently be in the form of a sterile solution or a lyophilized solid. In one embodiment, the formulation is present in the kit in lyophilized form. The term “lyophilized” has the conventional meaning, i.e. a freeze-dried composition, one which may be prepared in a sterile manner. In some embodiments, the kit comprises a biocompatible carrier for reconstituting the components in solvated or suspension form for administration, such as by injection, to a subject. In relation to the kit, the term “biocompatible carrier” and examples thereof are as defined above for the radiopharmaceutical composition.

The fibrinogen formulation may be contained in one suitable container or multiple containers. For instance, one container may include fibrinogen, a reducing agent, such as stannous chloride, and at least one chelating agent, such as DTPA dianhydride, p-SCN-Bz-DTPA, or HYNIC. A tonicity agent, such as sodium chloride may be included in the first container. In another embodiment, the kit may include at least two containers. For instance, a first container may contain fibrinogen and at least one chelating agent, such as DTPA dianhydride, p-SCN-Bz-DTPA, or HYNIC; a second container may contain a reducing agent, such as stannous chloride. A tonicity agent, such as sodium chloride may be included in the first container, second container, or both the first and second containers. Alternatively, a tonicity agent, such as sodium chloride, may be included in a third container.

In yet another embodiment, the kit may include at least three containers. For instance, a first container may contain fibrinogen and at least one chelating agent, such as such as DTPA dianhydride, p-SCN-Bz-DTPA, or HYNIC; a second container may contain a reducing agent, such as stannous chloride; a third container may contain a biocompatible carrier, such as a sterile aqueous solution. A tonicity agent, such as sodium chloride may be included in the first container, second container, third container, or a combination thereof. Alternatively, a tonicity agent, such as sodium chloride, may be included in a fourth container.

In one embodiment, the kit comprises the following components in one container under nitrogen gas as a lyophilized powder:

| | |
|---|---|
| Fibrinogen | 1-15 mg, such as 10 mg |
| DTPA dianhydride | 0.36 μg-536.0 μg, such as 125.1 μg |
| Stannous Chloride | 0.001-1.90 mg, such as 0.89 mg |
| Sodium Dihydrogen Phosphate | 3.0 to 24.0 mg, such as 9.6-14.4 mg |
| Optionally, Sodium Chloride | 1.17-8.77 mg, such as 1.75-4.09 mg |
| Optionally, Sodium Ascorbate | 0.99-7.92 mg, such as 3.96 mg |
| pH on reconstitution | 7.0 to 8.4 |

| | |
|---|---|
| human fibrinogen (FIBRYGA ®) | 1-15 mg, such as 10 mg |
| p-SCN-Bz-DTPA | 0.65 μg-974.9 μg, such as 227.5 μg |
| Stannous Chloride | 0.001-1.90 mg, such as 0.89 mg |
| Sodium Dihydrogen Phosphate | 3.0 to 24.0 mg, such as 9.6-14.4 mg |
| Optionally, Sodium Chloride | 1.17-8.77 mg, such as 1.75-4.09 mg |
| Optionally, Sodium Ascorbate | 0.99-7.92 mg, such as 3.96 mg |
| pH on reconstitution | 8.0 to 8.4 |

In another embodiment, the it comprises the following components in two containers under nitrogen gas as lyophilized powders:

| | |
|---|---|
| Container 1: | |
| Fibrinogen | 1-15 mg, such as 10 mg |
| DTPA dianhydride | 0.36 μg-536.0 μg, such as 125.1 μg |
| Sodium Dihydrogen Phosphate | 3.0 to 24.0 mg, such as 9.6-14.4 mg |
| Optionally, Sodium Chloride | 1.17-8.77 mg, such as 1.75-4.09 mg |
| Optionally, Sodium Ascorbate | 0.99-7.92 mg, such as 3.96 mg |
| Container 2: | |
| Stannous Chloride | 0.001-1.90 mg, such as 0.89 mg |
| Optionally, Sodium Chloride | 1.17-8.77 mg, such as 1.75-4.09 mg |
| Optionally, Sodium Ascorbate | 0.99-7.92 mg, such as 3.96 mg |
| pH on reconstitution | 7.0 to 8.4 |

| | |
|---|---|
| Container 1: | |
| human fibrinogen (FIBRYGA ®) | 1-15 mg, such as 10 mg |
| p-SCN-Bz-DTPA | 0.65 μg-974.9 μg, such as 227.5 μg |
| Sodium Dihydrogen Phosphate | 3.0 to 24.0 mg, such as 9.6-14.4 mg |
| Optionally, Sodium Chloride | 1.17-8.77 mg, such as 1.75-4.09 mg |
| Optionally, Sodium Ascorbate | 0.99-7.92 mg, such as 3.96 mg |
| Container 2: | |
| Stannous Chloride | 0.001-1.90 mg, such as 0.89 mg |
| Optionally, Sodium Chloride | 1.17-8.77 mg, such as 1.75-4.09 mg |
| Optionally, Sodium Ascorbate | 0.99-7.92 mg, such as 3.96 mg |
| pH on reconstitution | 7.0 to 8.4 |

In some embodiments, the kit further comprises additional components for the purification of the radiopharmaceutical composition. For instance, the kit may include components for the gel filtration of the composition. In one embodiment, the kit includes a G-25 Sephadex column in an appropriate size for purification of the radiolabeled fibrinogen composition. In another embodiment, the kit includes a container with a suitable elution buffer for the gel filtration of the radiolabeled fibrinogen composition. In yet another embodiment, the kit may contain a filter suitable for the sterile filtration of the radiolabeled fibrinogen composition. Membrane filter media include, but are not limited to, Polyvinylidene difluoride (PVDF), Polyether sulfone (PES), Cellulose acetate (CA), mixed cellulose ether (MCE), regenerated cellulose, polytetrafluoroethylene (PTFE), and nylon. In one embodiment, the kit includes a 0.22 micron pore size PVDF membrane filter.

In some embodiments, the kit further comprises instructions for handling, storing, and/or using the components.

### Unit Dose and Method of Preparation

Also disclosed is a unit dose of the radiopharmaceutical ^{99m}Tc-fibrinogen which comprises the radiopharmaceutical composition of the invention, having a ^{99m}Tc radioactive content suitable for imaging a single subject. The term “unit subject dose” or “unit dose” means a ^{99m}Tc-fibrinogen radiopharmaceutical composition having a ^{99m}Tc radioactive content suitable for in vivo imaging after administration to a single subject.

The unit subject dose is provided in a sterile form suitable for human administration in a suitable container or syringe. Such syringes are suitable for clinical use and may be disposable so that the syringe would only ever be used with an individual subject. The syringe may optionally be provided with a syringe shield to protect the operator from radiation dose. Suitable such radiopharmaceutical syringe shields are commercially available and may comprise either lead or tungsten. The unit dose of ^{99m}Tc-fibrinogen radiopharmaceutical may alternatively be provided in a container which has a seal which is suitable for puncturing with a hypodermic needle (e.g. a crimped-on septum seal closure). The ^{99m}Tc radioactive content of the unit dose may be from 10-20 mCi or from 5-50 mCi.

Also disclosed is a process for the preparation of one or more unit subject doses of the radiopharmaceutical ^{99m}Tc-fibrinogen, which comprises: (i) reconstituting the disclosed kit with either a sterile solution of ^{99m}Tc-pertecnetate or first a biocompatible carrier followed by a sterile solution of ^{99m}Tc-pertecnetate; (ii) optionally carrying out step (i) in the presence of an antimicrobial preservative; (iii) allowing ^{99m}Tc-fibrinogen complex formation to take place; optionally checking the radiochemical purity of the ^{99m}Tc-fibrinogen complex; withdrawing a unit dose from the solution of step (iii) into a suitable syringe or container; and optionally repeating step (v) with an additional syringe or container at later times to give further unit doses. The process may be carried out in the absence of an antimicrobial preservative. The sterile solution of ^{99m}Tc-pertechnetate may be obtained from a technetium generator.

### Imaging Methods

The disclosed fibrinogen radiopharmaceutical compositions are useful in the detection of internal bleeding in a subject. Accordingly, also disclosed are methods for radioactive imaging in which a detectable amount of the fibrinogen radiopharmaceutical composition is administered to a subject and a region of interest of the subject is imaged.

Various techniques may be used to administer the composition to the subject, including, for example, parenteral injection or infusion, such as intravenous (i.v), intramuscular (i.m.), intracutaneous, intradermal, subcutaneous (s.c., s.q., sub-Q, Hypo), intraperitoneal (i.p.), intra-arterial, intramedullary, intracardiac, intra-articular (joint), intrasynovial (joint fluid area), intracranial, intraspinal, and intrathecal (spinal fluids). In one embodiment, the fibrinogen radiopharmaceutical composition is administered by intravenous injection. Any known device useful for parenteral injection or infusion of drug formulations can be used to effect such administration.

Imaging of the subject may be performed using known procedures. Appropriate detection methods include SPECT (single photo emission computed tomography), gamma detectors (gamma cameras), PET (positron emission tomography), radiography, autoradiography, or a combination thereof.

The compositions may be used to detect internal bleeding in various regions of interest in a subject, including, for example, stomach, small intestine, large intestine (i.e. colon), esophagus, head, neck, torso, extremities, trachea, lungs, mediastinum, pleural space, liver, spleen, kidneys, pancreas, gallbladder, retroperitoneum, pelvis, rectum, gastrointestinal system, or a combination thereof. In one embodiment, the region of interest is within the gastrointestinal system of the subject. In yet another embodiment, the region of interest is selected from the group consisting of stomach, small intestine, large intestine, esophagus, and a combination thereof.

As noted herein, the presently-disclosed subject matter can be used to identify and localize both active and inactive GI bleeds in either upper or lower regions of the GI tract. Additionally, the presently-disclosed subject matter can be used in localizing other, non-GI sources of internal bleeding.

For example, additional application of the presently-disclosed subject matter include the detection of the bleeding site for subjects presenting with hematuria and/or as an effective tool for differential diagnosis of subjects presenting with hematuria. Hematuria, or blood in the urine, is a symptom that can result from malignant and non-malignant causes, including kidney disease, kidney cancer, urinary tract infection, bladder cancer, prostatic hyperplasia, prostate cancer, medications, etc. [8-11].

For another example, the presently-disclosed subject matter could be used for diagnosing, localizing, and monitoring stroke, such as for localizing thrombi to inform and guide endovascular interventions.

Embodiments of the presently-disclosed subject matter are also set forth in the Examples and in the Claims herein below.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1. Preparation of ^{99m}Tc-DTPA-DA-Fibrinogen Using Stannous Chloride Reducing Agent

Materials used were as follows. lyophilized human fibrinogen containing glycine, sodium citrate dihydrate, L-arginine hydrochloride, sodium chloride as excipients (FIBRYGA®) was obtained from Octapharma. Diethylene triamine penta-acetic acid (DTPA) dianhydride and stannous chloride dihydrate were purchased from Sigma-Aldrich Chemical Co. and used without further purification. PD-10 column (GE Healthcare) was preconditioned with 0.1M PBS buffer (8 mL). 0.1M NaHCO₃ solution was diluted 10 mL of 8.4% of NaHCO₃ into 90 mL of DI water. 0.1M PBS buffer with 0.15M NaCl, pH 8.0 was made by NaCl 0.8024 g; KCl 0.2100 g; Na₂HPO₄·2H₂O 1.8198 g; KH₂PO₄ 0.2451 g dissolved in 100 mL of DI H₂O, adjusted pH=8 by adding 0.2 mL of 4M NaOH solution. Fibrinogen solution was dissolved 47.52 mg of lyophilized human fibrinogen (FIBRYGA®) containing 16.71 mg of fibrinogen in 1.5 mL of 0.1M of NaHCO₃. 25 M of DTPA-dianhydride solution was dissolved 9.18 mg of DTPA-dianhydride in 1020 μL of anhydrous DMSO. Fresh stannous chloride dihydrate in 0.1N HCl: 19.76 mg was dissolved in 10 mL of 0.1N HCl solution.

Conjugated fibrinogen was prepared and purified as follows. An aliquot of 125 L DTPA-dianhydride (25 M) was added to 1.5 mL of Fibrinogen solution with thorough stirring at room temperature. pH 9.0; 15 min later another 125 μL aliquot of DTPA-dianhydride was added with constant stirring. After 1 hr incubation, DTPA-DA-Fibrinogen was separated by gel chromatography on a preconditioned PD-10 column by eluting with 0.1M PBS buffer. Each fraction was collected by 15 drops. The protein content of the purified DTPA-DA-fibrinogen was confirmed with iodine stain.

The DTPA-DA-Fibrinogen was radiolabeled with ^{99m}Tc as follows. A stannous chloride stock solution (25 L) was added to 0.6 mL of purified DTPA-DA-fibrinogen (fraction6). 0.5 mL of ^{99m}Tc-pertechnetate solution (˜20 mCi) was immediately added. The resultant solution was incubated at room temperature for 20 min. The pH of the final mixture was 7.7. The crude product of ^{99m}Tc-DTPA-DA-Fibrinogen was tested by thin lay chromatography (TLC).

^{99m}Tc-DTPA-DA-Fibrinogen was purified as follows. The crude ^{99m}Tc-DTPA-DA-Fibrinogen was loaded on a preconditioning of PD-10 column and eluted the column with 0.1M PBS buffer to remove any of the impurities of the radio activities. Each fraction collected 0.5 mL˜1.0 mL.

Thin Lay Chromatography was conducted as follows. The labeling efficiencies with 99mTc were chromatographically evaluated by using ITLC-SG paper strip developed with acetone, saline and ethanol:ammonium:water (2:1:5) solvent system. The paper strips were counted by imaging in Bioscan AR2000 Radio-TLC Imaging scanner. Typically, the crude production of 99mTc-DTPA-DA-Fibrinogen was more than 80% and purity of final product was more than 95% after purification.

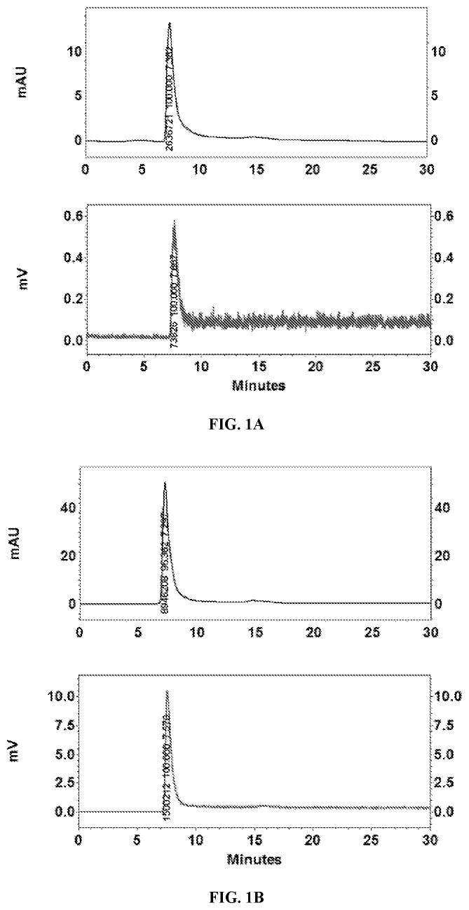

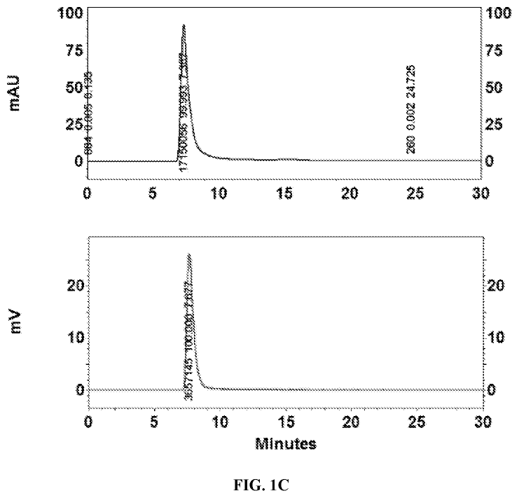

High Performance Liquid Chromatography (HPLC) was conducted. The labeling purity of ^{99m}Tc-DTPA-DA-fibrinogen was determined by size exclusion HPLC. 20˜50 μL of the purified ^{99m}Tc-DTPA-DA-Fibrinogen solution was injected and chromatography was performed with a size exclusion HPLC column (Superdex, 200 Increase, 10/300 GL, Code: 17-5174-01, Lot: 10041069) eluted isocratically with 14× PBS (5×) buffer at 1 mL/min flow-rate and monitored by UV absorbance at 280 nm and Eckert & Ziegler radioactivity detector using a Hitachi HPLC system. Results are provided in FIGS. 1A-1C.

The stability of ^{99m}Tc-DTPA-DA-fibrinogen was evaluated for 5 hours by TLC. There were not any changes within 5 hours.

### Example 2: High-Efficiency Protocol to Produce Biologically Active [^{99m}Tc]-Fibrinogen

Human fibrinogen concentrate containing human albumin, L-arginine hydrochloride, sodium chloride, sodium citrate and optionally pH adjusters sodium hydroxide/hydrochloric acid as excipients (RIASTAP®) was obtained and reconstituted with sterile water for injection (WFI). The vial was then gently swirled periodically until the powder was completely dissolved. The reconstituted solution was aliquoted into 5 mL vials for subsequent re-lyophilization in a Virtis AdvantagePlus EL freeze dryer using the following recipe. The samples were frozen at −45° C. on a shelf freezer followed by a 60 min hold at that temperature. After the freezing step, the condenser was adjusted to −85° C. and held at that temperature throughout the run. The pressure was adjusted to 100 mtorr using a Leybold Trivac E2 Dual-stage rotary vane vacuum pump and held at that pressure throughout the entire run. The shelf temperature was held at −45° C. for an additional 120 minutes, followed by an increase to −10° C. over 60 minutes. The shelf temperature was held at −10° C. for 1,200 minutes, increased to 10° C. over 60 min and then held at 10° C. for at least an additional 180 minutes. After this final step the samples were stoppered under nitrogen, removed from the lyophilizer, capped, and then stored under refrigerated conditions for further use.

The re-lyophilized sample was reconstituted with 0.95 mL sterile irrigation water and mixed well to make a solution with a concentration of −23 mg/mL. A fresh stock solution of 10 mM SnCl₂·2HO (˜2 mg/mL) in 0.1N HCl was prepared immediately prior to radiolabeling. The pH of the fibrinogen solution was adjusted with 0.1N Na₂CO₃. SnCl₂·2H₂O stock was added to the Fibrinogen solution followed immediately by addition of Na ^{99m}TcO₄ solution (0.5-3 mL) (pH 7.5-8).

To determine the optimal reaction conditions, a number of reaction variables were tested including fibrinogen mass, tin (II) chloride (SnCl₂) mass, types of solvents/buffers (pH), and the reaction time as described below. The radiolabeling yield was determined by radio-TLC using the following two systems:
System 1: Acetone/ITLC-SG (Rf=0: hydrolyzed ^{99m}TcO₂ and ^{99m}Tc-Fibrinogen; Rf=1.0: free ⁹⁹TcO₄⁻)
System 2: EtOH:NH₄OH:H₂O=2:1:5/ITLC-SG (Rf=0: hydrolyzed ^{99m}TcO₂; Rf=1.0: ^{99m}Tc-Fibrinogen and free ^{99m}TcO₄⁻)

$$Radiolabeling yield = \frac{\begin{matrix}CPM Rf 0. [ CPM ( {}^{99 m} Tc - Fibrinogen + {}^{99 m} {Tc O}_{2} in system 1 ) ] - \\ CPM Rf 0. ( {CPM}^{99 m} {Tc O}_{2} in system 2 )\end{matrix}}{CPM total in system 1}$$

$$or$$

$$Radiolabeling yield = \frac{\begin{matrix}CPM Rf 1. [ CPM ( {}^{99 m} Tc - Fibrinogen + {}^{99 m} Tc {O}_{4}^{-} in system 2 ) ] - \\ CPM Rf 1. ( {CPM}^{99 m} {Tc O}_{4}^{-} in system 1 )\end{matrix}}{CPM total in system 2}$$

The initial reaction variable tested was reaction time of radiolabeling with ^{99m}Tc (Table 1). The experimental conditions were 0.5 mL of Fibrinogen solution (11.5 mg), 50 μL 0.1M Na₂CO₃, 50 μL of SnCl₂·H₂O (100 μg) and 2 mL of Na^{99m}TcO₄ solution (7.98 mCi, pH 7.5-8).

TABLE 1
Optimized Labeling Time for Radiolabeling Fibrinogen with ^{99m}Tc

| | | | | Label- |
|---|---|---|---|---|
| | | | EtOH:NH₄OH:H₂O = | ing |
| Time | | Acetone | 2:1:5 | yield |
| 10 | min | Bottom(Rf = 0): 100% | Bottom(Rf = 0): 0% | 100% |
| | | Top(Rf = 1.0): 0% | Top(Rf = 1.0): 100% | |
| 1 | hr | Bottom(Rf = 0): 99.54% | Bottom(Rf = 0): 2.08% | 97.92% |
| | | Top(Rf = 1.0): 0.46% | Top(Rf = 1.0): 97.92% | |
| 4 | hrs | Bottom(Rf = 0): 100% | Bottom(Rf = 0): 1.38% | 98.62% |
| | | Top(Rf = 1.0): 0% | Top(Rf = 1.0): 98.62% | |
| 8 | hrs | Bottom(Rf = 0): 99.67% | Bottom(Rf = 0): 1.23% | 98.77% |
| | | Top(Rf = 1.0): 0.33% | Top(Rf = 1.0): 98.77% | |
| 24 | hrs | Bottom(Rf = 0): 98.97% | Bottom(Rf = 0): 1.72% | 97.25% |
| | | Top(Rf = 1.0): 1.03% | Top(Rf = 1.0): 98.28% | |

Next, the pH of the reaction solution was optimized (Table 2). The reaction conditions were 0.1 mL of Fibrinogen solution (2.3 mg), 15 μL, 22 μL, 30 μL and 35 μL of 0.1M Na₂CO₃ to obtain solutions with pH 8, 8.5, 9 and 9.2 respectively, 15 μL of SnCl₂·2H₂O (30 μg) and 0.5 mL of Na^{99m}TcO₄ solution (1.2-1.5 mCi).

TABLE 2
Optimized Reaction pH for Radiolabeling Fibrinogen with ^{99m}Tc

| pH | Acetone | EtOH:NH₄OH:H₂O = 2:1:5 | Lbl. yield |
|---|---|---|---|
| 8 | Bottom(Rf = 0): 99.55% | Bottom(Rf = 0): 16.85% | 82.7% |
| | Top(Rf = 1.0): 0.45% | Top(Rf = 1.0): 83.15% | |
| 8.5 | Bottom(Rf = 0): 92.30% | Bottom(Rf = 0): 25.42% | 66.88% |
| | Top(Rf = 1.0): 7.70% | Top(Rf = 1.0): 74.58% | |
| 9 | Bottom(Rf = 0): 90.84% | Bottom(Rf = 0): 29.82% | 61.02% |
| | Top(Rf = 1.0): 9.16% | Top(Rf = 1.0): 70.18% | |
| 9.2 | Bottom(Rf = 0): 89.90% | Bottom(Rf = 0): 27.53% | 62.37% |
| | Top(Rf = 1.0): 10.10% | Top(Rf = 1.0): 72.47% | |

The amount of Fibrinogen was optimized concurrently with the reaction time (Table 3). The reaction conditions were 2.5 mg, 5 mg, 8 mg, and 11.5 mg of Fibrinogen, 35 μL of 0.1M Na₂CO₃, 35 μL of SnCl₂·2H₂O (70 μg) and 3 mL of Na^{99m}TcO₄ solution (7-8 mCI).

TABLE 3
Optimized Amount of Fibrinogen for Radiolabeling Fibrinogen with ^{99m}Tc

| | | | | | | 11.5 mg | 11.5 mg |
|---|---|---|---|---|---|---|---|
| | | | | | | (100 μg | (30 μg |
| Fibrinogen | | 2.5 mg | 5 mg | 8 mg | 11.5 mg | SnCl₂•2H₂O) | SnCl₂•2H₂O) |
| 25 | min | 75.69% | 86.24% | 90.03% | 88.09% | 100% | 95.51% |
| 2 | hrs | 79.27% | 85.53% | 90.10% | 100% | 97.92% | 96.28% |
| 4 | hrs | | | | | 98.62% | 96.93% |
| 6 | hrs | 74.79% | 90.63% | 92.46% | 97.08% | | 97.06% |
| 8 | hrs | | | | | 98.77% | 96.69% |
| 24 | hrs | 63.99% | 89.87% | 88.85% | 95.59% | 97.25% | 54.71% |

Finally, the amount of SnCl₂-2HO was optimized concurrently with the reaction time (Table 4). The reaction conditions were 8 mg of Fibrinogen, 15 μL of 0.1M Na₂CO₃, 15 μL (30 μg), 25 μL (50 μg), 35 μL (70 μg), and 45 μL (90 μg) of SnCl₂·H₂O and 3 mL of Na^{99m}TcO₄ solution (7-8 mCi).

TABLE 4
Optimized Amount of SnCl₂•2H₂O
for Radiolabeling Fibrinogen with ^{99m}Tc

| | | | | |
|---|---|---|---|---|
| SnCl₂•2H₂O | 30 μg | 50 μg | 70 μg | 90 μg |

| | | | | | |
|---|---|---|---|---|---|
| 10 | min | 95.88% | 90.01% | 87.90% | 88.60% |
| 2 | hrs | 95.78% | 95.23% | 91.25% | 94.04% |
| 6 | hrs | 94.52% | 94.68% | 92.99% | 91.14% |
| 24 | hrs | 67.37% | 97.01% | 89.93% | 84.14% |

Optimized conditions were found and the following experimental protocol was found to be reproducible for the preparation of ^{99m}Tc-labeled Fibrinogen. The re-lyophilized sample described above was reconstituted with 0.95 mL sterile irrigation water and mixed well to make a solution with a concentration of ˜23 mg/mL. 0.5 mL of Fibrinogen solution (11.5 mg of Fibrinogen) was pipetted into a 10-mL sterile vial. A fresh stock solution of 10 mM SnCl₂·2H₂O (˜2 mg/mL) in 0.1N HCl was prepared immediately prior to radiolabeling. The pH of the fibrinogen solution was adjusted to pH˜9 by pipetting 25 μL of 0.1N Na₂CO₃ into the Fibrinogen solution. 25 μL of SnCl₂·2H₂O stock solution (50 g) was added to the Fibrinogen solution (pH 7.5-8) followed immediately by addition of Na ^{99m}TcO₄ solution (0.5-3 mL). The reaction is incubated for 30 minutes at room temperature. Some critical factors for optimizing radiolabeling yield include preparing the SnCl₂·2H₂O stock solution as close as possible to the radiolabeling time, adjusting the pH prior to addition of SnCl₂·2H₂O, and immediately adding Na^{99m}TcO₄ solution after addition of SnCl₂·2H₂O.

The stability of the ^{99m}Tc-labeled Fibrinogen was tested at room temperature for 24 hours, in vitro in rat serum at 37° C. for 24 hours and in vivo in rat urine. ^{99m}Tc-Fibrinogen was stable at room temperature for 24 hours based on lack of significant dissociation of isotope and protein as measured by radioactive TLC (see Tables 1, 3, and 4 above).

For analysis of ^{99m}Tc-Fibrinogen stability in rat serum, rat serum was obtained from the whole blood of normal rats by centrifugal force removal of red blood cells. The stability of the labeled ^{99m}Tc-Fibrinogen was measured by radio-TLC. The ^{99m}Tc-Fibrinogen was prepared as 83 mCi/0.5 mL. 10 μL and 20 μL aliquots of ^{99m}Tc-Fibrinogen were separately added into 200 μL of rat serum and incubated at 37° C. in a water bath for 24 hours. 20 μL of ^{99m}Tc-Fibrinogen was incubated with 200 μL of saline at the same conditions as a control. The final concentrations of Fibrinogen were calculated to be 436 g Fibrinogen/serum solution (1.959 mCi) and 218 g Fibrinogen/serum solution (1.08 mCi) for the 20 and 10 μL aliquots respectively. The final activity of the saline control was 1.979 mCi. Aliquots were removed from the reaction mixture after incubation for 0.5, 3, 6, and 24 hours at 37° C. The percentage of ^{99m}Tc-Fibrinogen was determined using TLC developed with acetone and EtOH/NH₄OH/H₂O=2/1/5. The results are shown in Table 5 and demonstrate that as compared with the control sample, desorption of free Tc-99m from Fibrinogen cannot be observed. More than 90% of the radioactivity was associated with Fibrinogen after 24 hours of incubation.

TABLE 5
TLC analysis of in vitro rat serum stability of ^{99m}Tc-Fibrinogen

| | | | Fibrinogen Concentration in | TLC radiolabeled |
|---|---|---|---|---|
| Time | | samples | serum solution | yield |
| 0.5 | hr | 10 μL | 218 μg Fib/serum mix | 97.68% |
| | | 20 μL | 436 μg Fib/serum mix | 91.71% |
| | | control | | 91.51% |
| 3 | hr | 10 μL | 218 μg Fib/serum mix | 91.65% |
| | | 20 μL | 436 μg Fib/serum mix | 96.89% |
| | | control | | 90.08% |
| 6 | hr | 10 μL | 218 μg Fib/serum mix | 94.14% |
| | | 20 μL | 436 μg Fib/serum mix | 97.99% |
| | | control | | 88.21% |
| 24 | hr | 10 μL | 218 μg Fib/serum mix | 88.39% |
| | | 20 μL | 436 μg Fib/serum mix | 92.68% |
| | | control | | 90.73% |

For the urine analysis, ^{99m}Tc-Fibrinogen was prepared as described above and was injected in the tail vein of healthy rats. The urine was collected 2 hours post-injection and was analyzed by radio-TLC. The results are shown in Table 6 and demonstrate no appreciable formation of ^{99m}Tc impurities.

TABLE 6
TLC analysis of urine collected 2 hours post tail vein injection of ^{99m}Tc-Fibrinogen in rats

| | Urine | | Aliquot | | Count | | | |
|---|---|---|---|---|---|---|---|---|
| Rat | estimated | Radioactivity | TLC | | time | R_{f} = 0 | R_{f} = 1.0 | Sample |
| # | (uL) | (mCi) | (uL) | eluent | (min) | (%) | (%) | name |
| 1 | 800 | 0.347 | 3 | System 1 | 5 | 100 | 0 | 170314_Ur1_1 |
| | | | 3 | System 2 | 5 | 0 | 100 | 170314_Ur1_3 |
| 2 | 600 | 0.270 | 3 | System 1 | 5 | 100 | 0 | 170314_Ur2_1 |
| | | | 3 | System 2 | 5 | 1.02 | 98.98 | 170314_Ur2_2 |
| 3 | 800 | 0.184 | 3 | System 1 | 10 | 100 | 0 | 170314_Ur3_1 |
| | | | 3 | System 2 | 10 | 0.73 | 99.27 | 170314_Ur3_2 |
| 4 | 50 (Na^{99m}TcO₄) | 0.0109 | 3 | System 1 | 10 | 2.68 | 97.32 | 170314_Ur4_1 |
| | | | 3 | System 2 | 10 | 0 | 100 | 170314_Ur4_3 |
| 5 | Not obtained | — | — | — | — | — | — | — |
| | | | — | — | — | — | — | — |

As set forth in this example a synthetic method has been established that reliably gives >90% labeling efficiency with <10% variation between labeling reactions. The probe shows excellent stability up to 24 hours both at room temperature and in rat serum at 37° C. and in urine at 2 hours post-injection. After incubation of ^{99m}Tc-Fibrinogen in rat serum at 37° C. for 24 hours, greater than 90% radioactivity remained associated with fibrinogen. Thus, this method reliably generates high quality [^{99m}Tc]-fibrinogen of acceptable specific concentration, stability and bioactivity.

### Example 3: Demonstrate the Utility and Efficacy of [^{99m}Tc]-Fibrinogen in an Animal Model of GI Bleeding

The process identified in Example 2 was used to produce an injectable SPECT tracer, to localize bleeding in a rat model. The in vivo efficacy of ^{99m}Tc-Fibrinogen was assessed using a colonic injury model. The proposed injury was made by performing a biopsy using a small animal endoscope (Karl Storz Veterinary Endoscopy, Goleta, CA) with a 3Fr instrument channel which will cause bleeding of the colon.

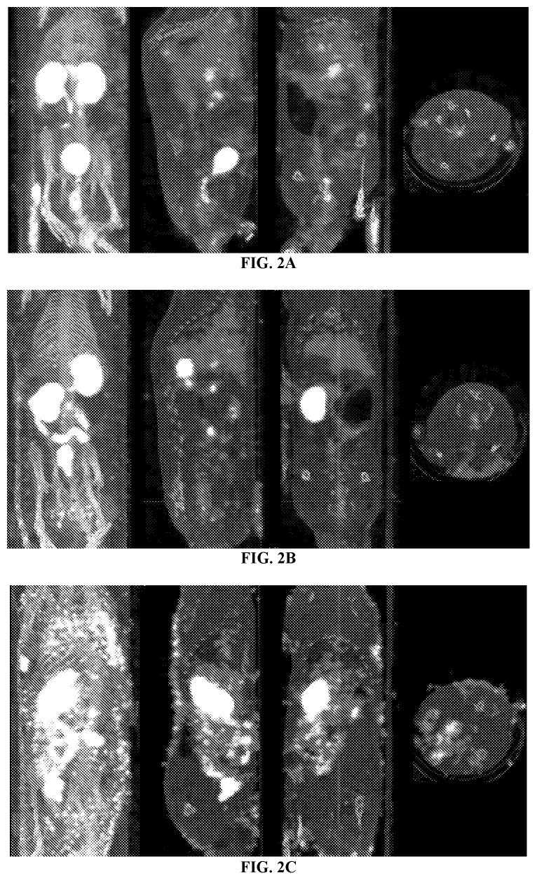

The first imaging study was conducted with six rats after colonic injury. Five rats received a tail vein injection of ^{99m}Tc-Fibrinogen and one received ^{99m}Tc—O₃ alone as a control. For two of the rats (Rats #1 and #2), high signal was detected at the sites thought to be the colonic injury at 30 minutes post-injection (FIGS. 2A, 2B). However, the injuries were too small to reliably conduct post-mortem analyses. The data from the remaining mice were either not able to be interpreted due to colon perforations (Rats #3 and #5) or had no signal at the injury site (Rat #6). No signal was observed at the injury site for the control compound (Rat #4, FIG. 2C). From this study, it was concluded that the useful imaging timeframe was within 30 minutes post-injection of tracer. High signal was observed in the kidneys, bladder and ureters, with no significant signal in the stomach, indicating that urinary excretion was the primary mode of elimination. However, significant challenges were observed with the colonic biopsy injury model including difficulty in visualizing very small biopsy injuries in rat colon post-mortem, lack of reproducibility of biopsy injury and unintended colonic perforations. Thus, the colonic biopsy injury model was replaced with the gastric injury model in future experiments.

The remaining imaging studies were performed using a rat gastric injury model. Rats were anesthetized with 2% isofluorane and were injected with ˜37 MBq of ^{99m}Tc-Fibrinogen or pertechnetate (^{99m}TcO₄) as a control compound via the tail vein a few minutes before injury. Approximately 60 minutes post radiotracer administration, the rats were imaged in a NanoSPECT/CT (Siemens Bioscan, Washington DC) with 24 projections and 60 seconds per projection followed by CT for a total imaging time of about 30 minutes. Then the rats were euthanized and tissue was harvested and counted for radioactivity in a gamma counter (Capintec, Florham Park, NJ).

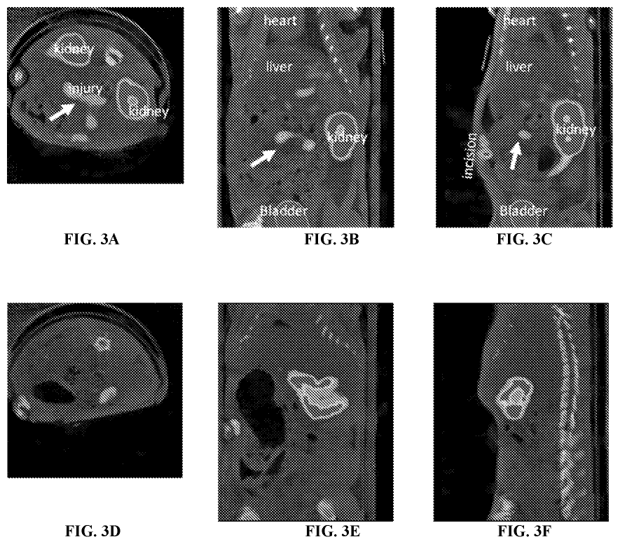

To compare the binding and accumulation in the injury site of the targeted agent versus a nonspecific control, five rats with gastric injury were imaged with either ^{99m}Tc-Fibrinogen or ^{99m}TcO₄. Three rats received a tail vein injection of ^{99m}Tc-Fibrinogen and two rats received ^{99m}TcO₄ as a control. Representative images are shown in FIGS. 3A-3F. The in vivo accumulation was quantified by drawing regions of interest (ROIs) around the major organs (liver, kidneys, heart, bladder), the incision site, the gastric injury site and muscle. The radioactivity obtained from the ROI was normalized to the injected dose (ID) and the values were expressed as % ID/gram (Table 7). In addition, the organ/muscle ratios were determined (Table 8). Ex vivo analysis was performed to confirm the SPECT/CT imaging results. The radioactivity was counted in the liver, kidneys, gastric injury site and a control healthy area. The activity/gram was calculated for each organ (Table 9) and the signal-to-noise ratio was calculated for the gastric injury in comparison to the control area (Table 10). From the SPECT images, it can be seen that ^{99m}Tc-Fibrinogen accumulated in the injury site while little signal could be detected in the injury site with ⁹⁹TcO₄. High signal was again observed in the kidneys and bladder. The quantitative data demonstrate that ^{99m}Tc-Fibrinogen specifically binds to the site of bleeding at the gastric injury site. Signal-to-noise (organ/muscle) ratios for the gastric injury site ranged from 11-16:1, an acceptable ratio at this stage of development. The in vivo imaging results were confirmed ex vivo. A higher signal-to-noise ratio was observed for the mice imaged with ^{99m}Tc-Fibrinogen in comparison to those imaged with ^{99m}TcO₄.

TABLE 7
Analysis of % ID/g for ^{99m}Tc-Fibrinogen
in Gastric Injury Rat Model

| ROI | Rat #1 | Rat #2 | Rat #3 |
|---|---|---|---|
| Kidney | 26.77 | 30.75 | 19.72 |
| Bladder | 136.93 | 121.13 | |
| Liver | 3.40 | 3.85 | 2.46 |
| Injury | 8.98 | 6.29 | 4.71 |
| Incision | 9.43 | 5.73 | 5.13 |
| Heart | 1.51 | | 1.86 |
| Muscle | 0.58 | 0.56 | 0.31 |

TABLE 8
Analysis of Organ/muscle ratios for ^{99m}Tc-
Fibrinogen in Gastric Injury Rat Model

| Tissue | Rat #1 | Rat #2 | Rat #3 |
|---|---|---|---|
| Kidney | 46.46 | 54.65 | 63.51 |
| Bladder | 237.66 | 215.28 | |
| Liver | 5.90 | 6.84 | 7.92 |
| Injury | 15.58 | 11.19 | 15.16 |
| Incision | 16.37 | 10.19 | 16.50 |
| Heart | 2.63 | | 5.98 |

TABLE 9
Ex vivo Analysis of Activity/gram for ^{99m}Tc-Fibrinogen
and ^{99m}TcO₄ in Gastric Injury Rat Model

| | Injected | Control | Gastric | | Kid- | Mucosal | Large |
|---|---|---|---|---|---|---|---|
| Rat # | compound | area | injury | Liver | ney | injury | bowel |
| 1 | ^{99m}Tc- | 2.3 | 17.4 | 9.8 | 157.3 | | |
| | Fibrinogen | | | | | | |
| 2 | ^{99m}Tc- | 9.9 | 20.9 | 18.1 | 125.7 | 26.4 | |
| | Fibrinogen | | | | | | |
| 3 | ^{99m}Tc- | 9.4 | 14.5 | 13.5 | 89.9 | 16.4 | |
| | Fibrinogen | | | | | | |
| 4 | ^{99m}TcO₄ | 22.4 | 4.4 | 2.6 | 2.6 | 4.5 | |
| 5 | ^{99m}TcO₄ | 11.7 | 5.9 | 1.2 | 1.0 | 12.6 | 1.4 |

TABLE 10
Ex vivo Analysis of Signal-to-noise ratios for ^{99m}Tc-
Fibrinogen and ^{99m}TcO₄ in Gastric Injury Rat Model

| | | Signal-to- |
|---|---|---|
| Rat # | Injected compound | noise ratio |
| 1 | ^{99m}Tc-Fibrinogen | 7.7 |
| 2 | ^{99m}Tc-Fibrinogen | 2.1 |
| 3 | ^{99m}Tc-Fibrinogen | 1.5 |
| 4 | ^{99m}TcO₄ | 0.2 |
| 5 | ^{99m}TcO₄ | 0.5 |

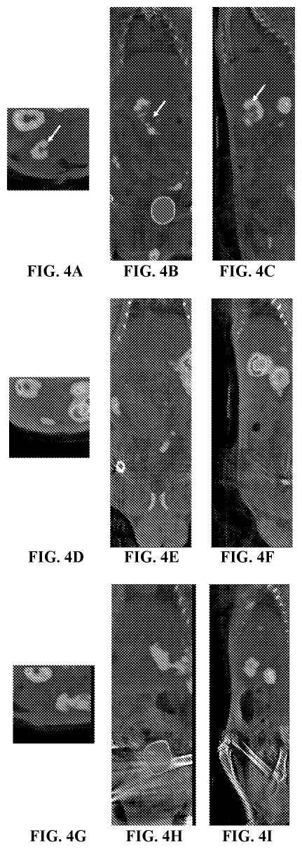

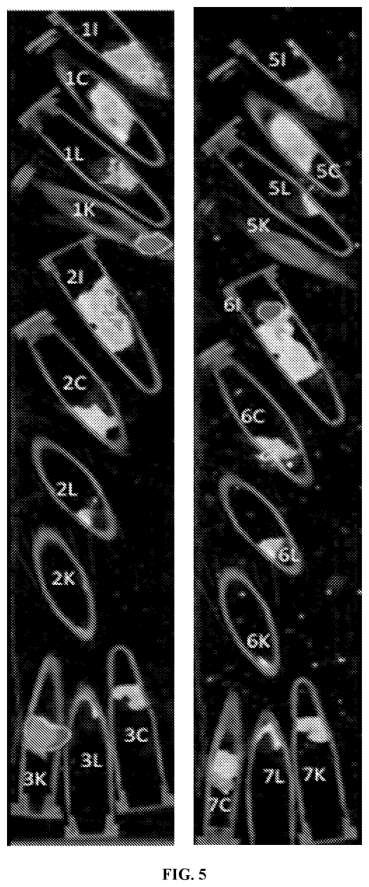

To test the specificity of the binding and accumulation of ^{99m}Tc-Fibrinogen to the injury site, ^{99m}Tc-Fibrinogen was used to image either normal rats (n=2) or rats following gastric injury (n=4) or sham injury (n=2). Representative images are shown in FIGS. 4A-4I. The in vivo accumulation was quantified by drawing regions of interest (ROIs) around the liver, kidneys, the surgery site, the gastric injury site and muscle. The radioactivity obtained from the ROI was normalized to the injected dose (ID) and the values were expressed as % ID/gram (Table 11). Ex vivo analysis was performed to confirm the SPECT/CT imaging results. The radioactivity was counted in the liver, kidneys, gastric injury site and a control healthy area. Images are shown in FIG. 5, the calculated concentration for each organ is given in Table 12, and the signal-to-noise ratio for the gastric injury in comparison to the control area is given in Table 13. From the SPECT images, it can be seen that ^{99m}Tc-Fibrinogen accumulated in the injury site. The quantitative data demonstrate that ^{99m}Tc-Fibrinogen binds to the site of bleeding at the gastric injury site. However, signal was also observed in the mice with sham injuries and one of the normal mice. From the ex vivo radioactivity counts, a higher signal was observed for the injury site compared to a control healthy site for all of the mice with gastric injuries.

TABLE 11
Analysis of % ID/g for ^{99m}Tc-Fibrinogen in Rats
with Gastric Injury, Sham Injury or Normal Rats

| Rat # | Treatment | Injury | Surgery | Liver | Kidney | Muscle |
|---|---|---|---|---|---|---|
| 1 | Gastric | 7.81 | 1.71 | 2.13 | 12.28 | 0.11 |
| | injury | | | | | |
| 2 | Gastric | 6.84 | 1.70 | 2.06 | 11.72 | 0.13 |
| | injury | | | | | |
| 3 | Sham | 9.92 | 1.39 | 2.51 | 14.26 | 0.06 |
| | injury | | | | | |
| 4 | Normal | | | 2.40 | 13.44 | 0.11 |
| 5 | Gastric | 4.42 | 2.23 | 1.91 | 12.10 | 0.17 |
| | injury | | | | | |
| 6 | Gastric | 6.83 | 2.46 | 1.78 | 10.55 | 0.28 |
| | injury | | | | | |
| 7 | Sham | 7.76 | 3.25 | 2.69 | 13.41 | 0.24 |
| | injury | | | | | |
| 8 | Normal | 9.54 | | 2.91 | 13.10 | 0.23 |

TABLE 12
Ex vivo Analysis of Concentration for ^{99m}Tc-Fibrinogen
in Rats with Gastric Injury or Sham Injury

| Rat # | Region | Treatment | Concentration |
|---|---|---|---|
| 1 | Injury | Gastric injury | 7.92 |
| 1 | Control | Gastric injury | 2.94 |
| 1 | Liver | Gastric injury | 6.57 |
| 1 | Kidney | Gastric injury | 13.29 |
| 2 | Injury | Gastric injury | 10.15 |
| 2 | Control | Gastric injury | 2.21 |
| 2 | Liver | Gastric injury | 4.80 |
| 3 | Control | Sham injury | 1.84 |
| 3 | Kidney | Sham injury | 15.50 |
| 5 | Injury | Gastric injury | 7.69 |
| 5 | Control | Gastric injury | 3.89 |
| 6 | Injury | Gastric injury | 13.93 |
| 6 | Control | Gastric injury | 5.00 |

TABLE 13
Ex vivo Analysis of Signal-to-noise ratios for
^{99m}Tc-Fibrinogen in Rats with Gastric Injury

| Rat # | Signal-to-noise ratio |
|---|---|
| 1 | 2.69 |
| 2 | 4.59 |
| 5 | 1.98 |
| 6 | 2.79 |

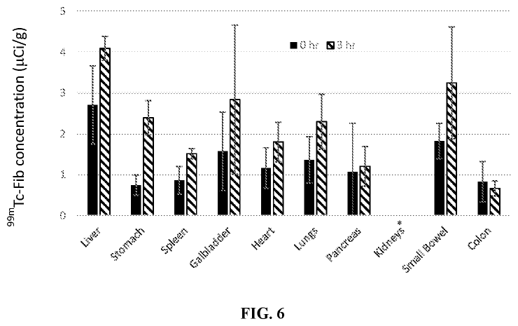

Finally, to measure the uptake of ^{99m}Tc-Fibrinogen by different organs and tissue types a biodistribution experiment was performed in normal healthy rats. A group of rats (n=4) were anesthetized with 2% isofluorane and injected with ˜37 MBq of ^{99m}Tc-Fibrinogen immediately upon arrival. The rats remained under anesthesia for 60 minutes. Then the rats were euthanized and tissues (liver, stomach, spleen, gallbladder, heart, lungs, pancreas, kidneys, small bowel, and colon) were harvested and counted for radioactivity in a Hidex AMG Automatic Gamma Counter (Lablogic, Brandon, FL) (FIG. 6, black bars). The integrity of the radiotracer was also tested. Approximately 3 hours after arrival of the radiotracer, a second group of rats (n=3) were anesthetized with 2% isofluorane and injected with ˜37 MBq of ^{99m}Tc-Fibrinogen. The cut & count experiment was repeated as above (FIG. 6, hatched bars). For most of the organs there is no significant difference in uptake whether the radiotracer is injected immediately upon arrival or 3 hours after arrival. However, the stomach shows significantly higher signal when the radiotracer is injected 3 hours after arrival compared to immediately after arrival.

These results establish the efficacy of a ^{99m}Tc-Fibrinogen tracer to locate the site of gastric injury in a rat model using SPECT/CT imaging. The in vivo imaging results were confirmed by ex vivo radiometric analysis. A higher signal was observed for the injury site compared to a control healthy site for all of the mice with gastric injuries. In addition, a higher signal-to-noise ratio was obtained for ^{99m}Tc-Fibrinogen in comparison to ^{99m}TcO₄, a nonspecific control. However, ^{99m}Tc-Fibrinogen signal was also observed in mice with sham injuries. Furthermore, high signal was observed in the kidneys/bladder indicating rapid renal clearance.

Thus, the results show that the ^{99m}Tc-Fibrinogen tracer binds to the site of bleeding at the gastric injury site successfully localizing the injury with a signal-to-noise ratio ≥2 (in most cases) when comparing injured vs. healthy tissue in the same rat.

### Example 4: One Vial Kit for Preparing a ^{99m}Tc-Fibrinogen Radiopharmaceutical Composition

A lyophilized powder for preparing a suspension of radiolabeled fibrinogen is prepared as follows. Fibrinogen will be reconstituted in 0.1 M sodium bicarbonate, pH 8.0 to a final fibrinogen concentration of approximately 20 mg/mL. Next, 140 μL of diethylenetriaminepentaacetic acid (DTPA)-Anhydride stock solution dissolved in DMSO at 25 μM will be added per mL of Fibrinogen solution and then stirred for 15 minutes. A subsequent 140 μL aliquot of DTPA-Anhydride will then be added to the fibrinogen solution followed by stirring for an additional 45 minutes. The mixture will next be passed through a G-25 Sephadex column equilibrated with 0.1 M phosphate buffered saline, pH 8.0. The eluted fractions containing fibrinogen, including fibrinogen conjugated to DTPA (DTPA-DA-Fibrinogen), will then be pooled. The final solution will be adjusted to a total fibrinogen concentration of approximately 10 mg/mL by diluting with 0.1 M sodium phosphate, pH 8.0 buffer. A volume of stannous chloride from a 0.1 M stock solution in 0.1 N HCl will also be added to the dilution buffer so that the final concentration will be 5 mM. Sodium ascorbate may be added to a final concentration of 20 mM. The pH of the solution will then be adjusted to 7.7 with 0.1 M sodium hydroxide. The solution will then be passed through a sterile filter and filled into glass vials at volumes ranging from 1.0 to 10 mLs. Samples may then be lyophilized by first freezing the sample at −45° C., followed by primary drying at −45 and −10° C. for up to 24 hours with a chamber pressure of 100 mtorr. Secondary drying may then be performed by first increasing the temperature from −10 to 10° C. over 1 hour and then holding at 10° C. for an additional 3 hours at 100 mtorr. The vials containing the DTPA-DA-Fibrinogen with buffer, stannous chloride, and sodium ascorbate will be backfilled with nitrogen, stoppered, and sealed.

To form the suspension of radiolabeled fibrinogen for administration, the lyophilized powder is reconstituted with a solution of NaTcO₄ [10⁻⁸ to 10⁻⁶ M, or 1.7 to 169.9 μg/L] in normal saline [0.9% sodium chloride]. A 1.0 mL volume of NaTcO₄ solution (5-20 mCi) in normal saline is added per 10 mg of total fibrinogen in each vial and allowed to reconstitute at room temperature until the powder is completely dissolved and a clear to slightly opalescent solution is obtained. The solution is allowed to react for 20 minutes.

The efficiency and purity of the ^{99m}Tc-Fibrinogen conjugate is determined by Thin Layer Chromatography (TLC) using a radio-TLC Imaging scanner. The ^{99m}Tc-Fibrinogen conjugate is further purified with a G-25 sephadex column, followed by passage through a filter.

### Example 5. Preparation of ^{99m}Tc-DTPA-DA-Fibrinogen Using Stannous Chloride Reducing Agent

Initially studies were performed using human fibrinogen concentrate containing human albumin, L-arginine hydrochloride, sodium chloride, sodium citrate and optionally pH adjusters sodium hydroxide/hydrochloric acid as excipients (RIASTAP®). We found that this source contains significant amounts of albumin (˜50%). As a result, albumin was also being labeled with ^{99m}Tc, acting as a radiolabeled blood pool imaging agent. To address this issue, we refined our synthesis method utilizing non-GMP pure fibrinogen from Sigma that does not contain albumin. Fibrinogen from human plasma was obtained as a dry solid from Sigma-Aldrich (50-70% protein with >80% clottable protein, Lot #SLBT8666). The fibrinogen (32±3 mg) was slowly dissolved in 0.1M NaHCO₃ (2.3 mL). To this was added 100 μL of a DTPA-dianhydride solution in DMSO (12.5 μM) at a temperature of 29° C. After allowing to react for 15 minutes, a second aliquot (100 μL) of the DTPA solution was added. After an additional 1 hour, the DTPA-DA-fibrinogen was purified by size exclusion column (PD-10, elution with 0.1 M PBS). The fractions containing the desired DTPA-DA fibrinogen were pooled and to this was added 25 μL of freshly prepared SnCl₂ [10 mM SnCl₂·2H₂O (˜2 mg/mL)] in 0.1 N HCl, immediately followed by addition of the Na ^{99m}TcO₄ in saline (30-38 mCi, 0.5 mL). The incorporation was allowed to proceed at room temperature for 20 min and monitored for completion by iTLC (conditions below). The crude product was then purified by size exclusion column and the fractions with the highest radioactivity were collected. Purity and identity of the final product was confirmed by both iTLC and radio-HPLC. In all cases the radiochemical purity of the final products were greater than 95%. The radiolabeling yield was determined by radio-TLC using the following three systems:
System 1: Acetone/ITLC-SG (R_{f}=0: hydrolyzed ^{99m}TcO₂ and ^{99m}Tc-Fibrinogen; R_{f}=1.0: free ^{99m}TcO₄);
System 2: Saline (R_{f}=1.0: free ^{99m}TcO₄ and ^{99m}Tc-DTPA-DA; R_{f}=0: other impurities; System 3: EtOH:NH₄OH:H₂O=2:1:5/ITLC-SG (R_{f}=0: hydrolyzed ^{99m}TcO₂; R_{f}=1.0: ^{99m}Tc-Fibrinogen and free ^{99m}TcO₄).
   Our data demonstrate that utilizing the bifunctional chelating agent (DTPA-DA) significantly increased the yield and purity of the radiolabeled product from 23.43% to 99.62%.

Overall, in vivo stability of the Technetium-99m labeled Fibrinogen-DTPA-DA was determined using drug naïve blood drawn from the same species of rat. The blood was spun down to obtain the serum and the serum was inoculated with the pure drug and tested for metabolites with incubation over the course of 24 hours. The results of these studies showed decomposition of the drug over a 24-hour timepoint to varying degrees. Analysis of the serum spiked with drug showed full stability of the drug after 40 minutes, 20% loss after 2 hours and 45% loss after 24 hours. In summary, we have identified a synthetic method that reliably gives >95% labeling efficiency. We have performed the synthesis multiple times with <5% variation between labeling reactions. Our probe shows excellent stability up to 24 hours both at room temperature and in rat serum at 37° C. Thus, we have identified a synthetic method that reliably generates high quality [^{99m}Tc]-DTPA-DA-fibrinogen of acceptable specific concentration and stability.

### Example 6. ^{99m}Tc-Fibrinogen in an Animal Model of GI Bleeding

We performed two separate in vivo studies, the first study was a biodistribution study to evaluate uptake levels in various tissues. Rats were anesthetized using 2% isoflurane. They were then administered 1 mCi ^{99m}Tc-DTPA-DA-fibrinogen, produced as described in Example 5, via tail vein at a volume of 200 μL and flushed with 100 μL of saline. Rats were euthanized at 0.5, 1, 3, 5, and 7 hours post injection, tissues collected and activity measured using a Hidex AMG Automatic Gamma Counter (Lablogic, Brandon, FL). Activity was described as Ci/g of tissue. The highest level of activity is observed in the kidneys with the liver and bladder demonstrating lower but significant uptake. Minimal uptake is observed in the stomach, heart, lungs, intestines or spleen, suggesting renal excretion of the radioligand. Additionally, the 1- and 3-hour time points had the highest level of uptake in the kidneys.

The second set of studies involved SPECT/CT imaging. The initial studies were conducted in healthy, uninjured rats to determine the optimal dose and pretreatment time for imaging. Rats were anesthetized using 2% isoflurane. They were then administered ^{99m}Tc-DTPA-DA Fibrinogen, produced as described in Example 5, via tail vein at a volume of 200 μL and flushed with 100 μL of saline. Rats were then placed in a stereotaxic head restraint, placed in the Inveon SPECT/CT (Siemens), and a 30 minute SPECT acquisition was performed followed by a CT. The SPECT images were manually co-registered to the CT image using Amide software. Regions-of-interest (ROIs) were drawn around the kidneys and liver and Standard Uptake Values (SUV) were determined accounting for body weight and injected dose. The initial time course study design assessed 0.5, 1, 3, 5 and 7 hour pretreatment times with an injected dose of 1.4 mCi. A significant amount of uptake can be observed in the kidneys, which appears to peak at 3 hours post injection, which correlates well with the biodistribution data. As the pretreatment time did not significantly affect the uptake of the radioligand, we chose to utilize a 5 minute pretreatment time for all future imaging studies to minimize any potential interference of the kidney uptake when evaluating GI bleeds.

After determining the optimal pretreatment time, we next evaluated the effects of lowering the injected dose on radioligand uptake. These studies indicated that the dose of ^{99m}Tc-DTPA-DA Fibrinogen can be significantly lowered (from 1.4 mCi to 300 μCi) without impacting the uptake of the radioligand in either the kidney (SUV: 0.68 versus 0.7, respectively) or liver (SUV: 0.24 versus 0.26 respectively). Therefore, to minimize the radiation exposure, we used lower doses in the GI injury models outlined below. Finally, we conducted a control study using ^{99m}Tc-pertechnetate SPECT imaging. Rats were administered 250 μCi ^{99m}Tc-pertechnetate via tail vein at a volume of 200 μL and flushed with 100 μL of saline. The primary organ of uptake is the liver, unlike the uptake observed with ^{99m}Tc-DTPA-DA Fibrinogen (n=3, data not shown).

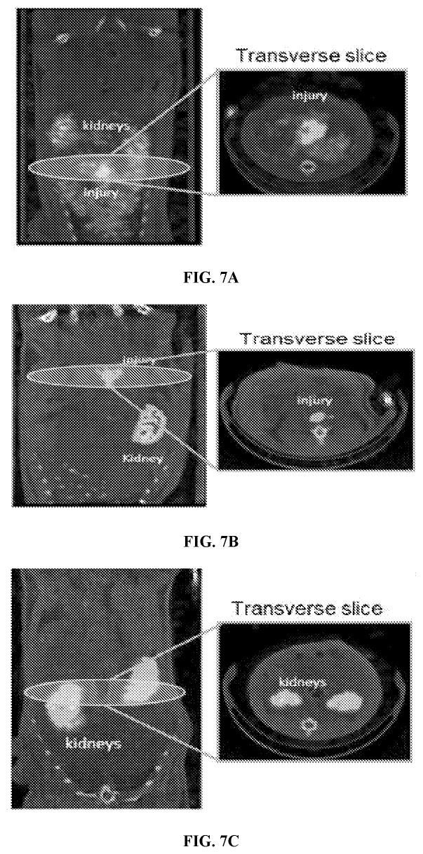

We used either the colonic injury, gastric mucosal stomach injury or sham surgery models to assess the in vivo efficacy of ^{99m}Tc-DTPA-DA Fibrinogen. For all studies, rats were anesthetized with 2% isoflurane and an incision made in the abdominal cavity. The gastric mucosal stomach injury was introduced by making a small incision in the stomach wall. Then a biopsy was taken from the inner mucosal wall of the stomach. To help localize the injury, the stomach at the site of the injury was tacked to the abdominal wall with sutures at the midline. The colonic injury was made by transecting the colon. The two transected ends of the colon were then tacked to the abdominal wall using sutures at the midline. The sham surgeries were conducted by making the same incision down the midline of the abdominal cavity and tacking either the uninjured stomach or colon to the abdominal wall. After all bleeding in the stomach or colon stopped, the abdominal cavity was closed. Rats were then injected with ˜250 μCi ^{99mTc}-DTPA-DA Fibrinogen, produced as described in Example 5, placed into the SPECT/CT, and a 30 minute SPECT scan followed by CT was acquired approximately 5 minutes after injection. The SPECT images were manually co-registered to the CT image using Amide software. Regions-of-interest (ROIs) were drawn around the injury (or uninjured organ for sham animals), kidneys, liver and muscle. Standard Uptake Values (SUV) were then calculated accounting for body weight and injected dose. The proposed metrics for success of the tracer were the ability of quantitative data from a SPECT/CT image to identify the injury site. FIG. 7 (n=3) displays representative images from the stomach (FIG. 7A) and colon injury model (FIG. 7B) versus a sham surgery (FIG. 7C). As can be seen in the images, significant ^{99m}Tc-DTPA-DA Fibrinogen uptake is observed at the site of the injury. No uptake is observed in the stomach in the sham surgery animals.

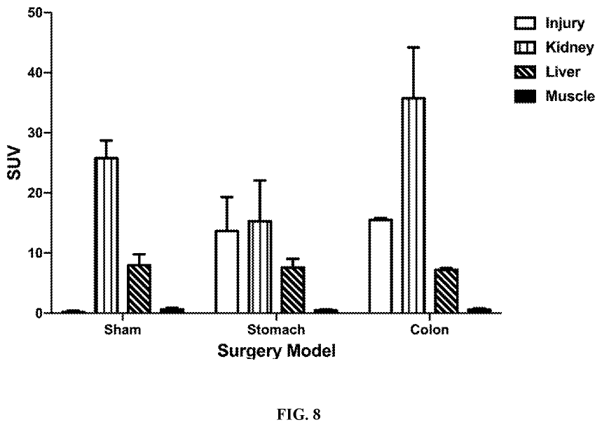

The quantified data from both injury models is depicted in FIG. 8 (n=2-3). No appreciable activity was measured in the sham surgery animals. Additionally, significant uptake is observed in both the stomach and colon injuries. The quantitative data demonstrate that ^{99m}Tc-Fibrinogen specifically binds to the site of injury. Following the conclusion of the imaging sessions, we confirmed the specific uptake in the injury site by direct excision of the injured tissue site. The excised tissue was rinsed of any blood using saline, placed in an eppendorf tube, and the radioactivity contained in this region was measured using SPECT imaging.

### Example 7. Bioactivity of Conjugated Fibrinogen

Fibrinogen samples and reference standards were prepared by reconstituting vials of Fibryga® (Human Fibrinogen, Lyophilized Powder for Reconstitution for Intravenous Use) with sterile water for injection (WFI) in a biological safety cabinet (Nuaire, serial number 72324 AER) at room temperature. The procedure included removing the cap from the vials and then cleaning the rubber stopper with an alcohol wipe. An Octajet® transfer device was then inserted through the stopper of the vial and followed by attachment of a vial containing 50 mLs of WFI. After transfer of WFI was completed, the vial was gently swirled periodically until the powder was completely dissolved and the solution was uniform (˜21 minutes). After the powder was completely reconstituted, the entire contents of the vial were transferred to a 50 mL polypropylene tube through a particle filter. Following filtration, 1 mL aliquots of the reconstituted solution were dispensed into 5 mL vials for subsequent re-lyophilization in a Virtis AdvantagePlus EL freeze dryer using the following recipe. Samples were frozen at −45° C. on a shelf freezer followed by a 60 min hold at that temperature. After the freezing step, the condenser was adjusted to −85° C. and held at that temperature throughout the run. The pressure was adjusted to 100 mtorr using a Leybold Trivac E2 Dual-stage rotary vane vacuum pump and held at that pressure throughout the entire run. The shelf temperature was held at −45° C. for an additional 120 minutes, followed by an increase to −10° C. over 60 minutes. The shelf temperature was held at −10° C. for 1,200 minutes, increased to 10° C. over 60 min, held at 10° C. for an additional 180 minutes, and then held at 20° C. for at least 180 minutes. After this final step the samples were stoppered under nitrogen, removed from the lyophilizer, capped, and then stored under refrigerated conditions for future use.

Fibrinogen was prepared for conjugation with DTPA-dianhydride or p-SCN-Bz-DTPA by first reconstituting 21 mg of the re-lyophilized sample with 2.0 mLs of normal saline under gentle swirling for ˜15-20 min at room temperature. A 0.5 mL aliquot was transferred to a polypropylene tube after reconstitution was completed for fibrinogen bioactivity testing (described below). The remaining 1.5 mLs of reconstituted sample was loaded onto a PD-10 column (Cytiva G-25 Sephadex; Cat No. 17085101) equilibrated with normal saline and eluted with the same solvent. The protein containing fractions were collected by measuring the UV absorbance at 280 nm, pooled into a 15 mL polypropylene tube, and then a 0.4 mL aliquot was transferred to another polypropylene tube for subsequent fibrinogen bioactivity testing. The pH of the remaining pooled fraction was adjusted to pH 9.0 by adding 0.1 mLs of 1.0 M sodium bicarbonate followed by titration with 0.1 and 1 N NaOH. One 0.4 mL aliquot from the pH adjusted mixture was removed for subsequent fibrinogen bioactivity testing and the remaining volume was split into three 0.4 mL aliquots for conjugation with DTPA-dianhydride or p-SCN-Bz-DTPA.

A calculation was made so that DTPA-dianhydride or p-SCN-Bz-DTPA was added to fibrinogen in 1:1, 5:1, and 10:1 molar ratios. In relation to DTPA-dianhydride, the protein concentration in the pH adjusted solution was 5.91 mg/mL, which was equivalent to ˜17.38 μM (using a molecular weight of 340,000 for Fibrinogen). Therefore, 6.95 nmols of fibrinogen was present in 0.4 mLs of solution (e.g. 17.38 μmol/L or 17.38 nmol/mL*0.4 mL). At this time DTPA-dianhydride (Sigma-Aldrich; Cat no. 284025-1G) was dissolved in anhydrous DMSO at 25 mM, which is 25 mmol/L, 25 μmol/mL, or 25 nmol/μL. The DTPA-dianhydride solution was then diluted to 1.74, 8.69, and 17.38 nmol/μL so that addition of 4 μL of each DTAP-dianhydride solution into 3 separate mixtures of fibrinogen would give a molar excess of 1:1, 5:1, and 10:1 (DTPA-dianhydride:Fibrinogen), respectively. Therefore, three separate solutions containing 6.95 nmols of fibrinogen with 6.95, 34.8, or 69.5 nmols of DTPA-dianhydride and a final DMSO concentration of ˜1% (v/v) were obtained. In relation to the p-SCN-Bz-DTPA, the protein concentration in the pH adjusted solution was 5.75 mg/mL, which was equivalent to ˜16.91 μM (using a molecular weight of 340,000 for Fibrinogen). Therefore, 6.76 nmols of fibrinogen was present in 0.4 mLs of solution (e.g. 16.91 μmol/L or 16.91 nmol/mL*0.4 mL). At this time p-SCN-Bz-DTPA (Macrocyclics, Cat no. B-305) was dissolved in a buffer composed of 50 mM sodium bicarbonate, pH 9.0 in saline to 5 mM, which was 5 mmol/L, 5 μmol/mL, or 5 nmol/μL. The p-SCN-Bz-DTPA solution was then diluted to 1 nmol/μL using the same buffer. To add a molar ratio of 1:1, 5:1, and 10:1 (p-SCN-Bz-DTPA:Fibrinogen), 6.8 μL of 1 nmol/μL and 6.8 and 13.5 μL of 5 nmol/μL p-SCN-Bz-DTPA, respectively, was added into 3 separate mixtures of fibrinogen, respectively. Therefore, three separate solutions containing 6.8 nmols of fibrinogen with 6.8, 33.8, or 67.8 nmols of p-SCN-Bz-DTPA were made. The samples were then incubated at room temperature for 45 minutes followed by purification of the Fibrinogen conjugates on a PD-10 column equilibrated with normal saline and eluted with the same. The protein containing fractions were collected as described above and then directly placed into a freezer at −80° C. The following morning the samples were thawed and then subjected to fibrinogen bioactivity testing.

The bioactivity for fibrinogen was determined using the turbidity assay to determine the concentration of clottable protein, which was normalized to the total protein concentration using the Biuret method <USP-1057>. The turbidity assay was based on the reference of Inada et al. (1978) Faster Determination of Clottable Fibrinogen in Human Plasma: An Improved Method and Kinetic Study, Clinical Chemistry, Volume 24-2, 351-353. The assay was conducted by adding 0.5 mL of the thawed Fibrinogen solution to 1.0 mL of reaction buffer (10 mM Tris-HCl, pH 7.0, 40 mM NaCl) and 0.1 mL of thrombin (Sigma-Aldrich, Cat No. T4648-10KU) solution in WFI (143 NIH units/mL) in a plastic cuvette, followed by immediate mixing of the solution with a pipette, and allowing the clot to develop for at least 20 minutes. The turbidity for each sample was measured at 450 nm in a Shimadzu UV-Vis 2600 Spectrophotometer against a blank sample, which had the same composition as the former except for the presence of thrombin. To assess the concentration of clottable fibrinogen in the DTPA-dianhydride:Fibrinogen conjugates, a calibration curve was generated from the absorbance values of 3.29, 2.29, 1.56, 1.13, 0.47, and 0.30 mg/ml fibrinogen reference standards diluted in the reaction buffer (10 mM Tris-HCl, pH 7.0, 40 mM NaCl). To assess the concentration of clottable fibrinogen in the p-SCN-Bz-DTPA:Fibrinogen conjugates, a calibration curve was generated from the absorbance values of 3.39, 2.40, 1.71, 1.29, 0.57, and 0.25 mg/mL fibrinogen reference standards diluted in the reaction buffer (10 mM Tris-HCl, pH 7.0, 40 mM NaCl).

In parallel to the turbidity assay, the total protein concentration using the Biuret method <USP-1057> was determined by adding 1 volume of sample to 1 volume of Biuret Reagent 1 (6% NaOH) followed by addition of 0.4 volumes of Biuret reagent 2 (3.46 g CuSO₄·5H₂O, 34.6 grams sodium citrate dihydrate, and 20 grams sodium carbonate per 200 mLs) relative to the sample volume. The mixture was allowed to stand for no less than 15 minutes and then the absorbance was measured at 545 nm using a Shimadzu UV-Vis 2600 Spectrophotometer within 90 minutes of addition of the Biuret reagent. Standard solutions were made using a human serum albumin (HSA) reference standard (Octapharma, Lot #C118STD08) to achieve the protein concentrations of 6.90, 6.21, 4.66, 2.33, 1.16, 0.58, 0.23, 0.09, and 0.05 mg/mL. The absorbance values were plotted vs the HSA concentration to generate a calibration curve, which was used to determine the total protein concentration of the fibrinogen reference standards and samples.

The fibrinogen bioactivity was determined by diluting each fibrinogen sample into the linear range of the turbidity assay and the Biuret method for reporting the clottable and total protein concentration in mg/mL. Next, the % bioactivity was calculated by dividing the clottable protein concentration by the total protein concentration and multiplying by 100 (equation 1).

$$\frac{\begin{matrix}Clottable protein concentration ( mg / mL ) * \\ ( dilution factor ) * 100\end{matrix}}{\begin{matrix}Total Protein concentration ( mg / mL ) * \\ ( dilution factor )\end{matrix}} = \% Bioactivity \qquad Equation 1$$

The fibrinogen activity of the DTPA-dianhydride:Fibrinogen conjugates was ˜85 and 88% for the normal saline reconstituted sample and for the pooled fractions from the first PD-10 column (Table 14). There was a slight drop in the fibrinogen bioactivity to 71% after pH adjustment to 9.0. During the pH step there was observed precipitate that went back into solution when NaOH aliquots were used to raise the pH to 9. The fibrinogen bioactivity of the recovered Fibrinogen conjugates was 101.3, 85.0, and 84.0% for the 1:1, 5:1, and 10:1 molar ratios, respectively.

TABLE 14
Summary of Fibrinogen Bioactivity using DTPA-Dianhydride

| | | Turbidity | Biuret | |
|---|---|---|---|---|
| | | Assay | Assay | |
| | | mg/mL | mg/mL | |
| Sample | | Clottable | Total | Bioactivity |
| No. | Fibrinogen Sample | Protein | Protein | % |
| 1 | Fibrinogen reconstituted in Normal | 10.70 | 12.62 | 84.8 |
| | Saline | | | |
| 2 | Fibrinogen collected from the PD-10 | 5.59 | 6.36 | 87.9 |
| | column in NS | | | |
| 3 | Fibrinogen adjusted to pH 9.0 with | 4.22 | 5.89 | 71.6 |
| | NaHCO₃ and NaOH | | | |
| 4 | DTPA-Dianhydride:Fibrinogen (1:1, | 1.53 | 1.51 | 101.3 |
| | mol:mol) Collected from PD-10 | | | |
| | column in NS | | | |
| 5 | DTPA- Dianhydride:Fibrinogen (5:1, | 1.25 | 1.47 | 85.0 |
| | mol:mol) Collected from PD-10 | | | |
| | column in NS | | | |
| 6 | DTPA- Dianhydride:Fibrinogen | 1.05 | 1.25 | 84.4 |
| | (10:1, mol:mol) Collected from PD- | | | |
| | 10 column in NS | | | |

Compared to the fibrinogen bioactivity using DTPA-Dianhydride, no significant loss of fibrinogen bioactivity was observed in the p-SCN-Bz-DTPA:Fibrinogen conjugates, as shown in Table 15.

TABLE 15
Summary of Fibrinogen Bioactivity using p-SCN-Bz-DTPA:Fibrinogen

| | | Turbidity | Biuret | |
|---|---|---|---|---|
| | | Assay | Assay | |
| | | mg/mL | mg/mL | |
| Sample | | Clottable | Total | Bioactivity |
| No. | Fibrinogen Sample | Protein | Protein | % |
| 1 | Fibrinogen reconstituted in Normal | 11.44 | 10.80 | 105.9 |
| | Saline | | | |
| 2 | Fibrinogen collected from the PD-10 | 6.47 | 6.02 | 107.5 |
| | column in NS | | | |
| 3 | Fibrinogen adjusted to pH 9.0 with | 5.21 | 5.75 | 90.6 |
| | NaHCO₃ and NaOH | | | |
| 4 | p-SCN-Bz-DTPA:Fibrinogen (1:1, | 1.36 | 1.38 | 98.6 |
| | mol:mol) Collected from PD-10 | | | |
| | column in NS | | | |
| 5 | p-SCN-Bz-DTPA:Fibrinogen (5:1, | 1.66 | 1.75 | 94.9 |
| | mol:mol) Collected from PD-10 | | | |
| | column in NS | | | |
| 6 | p-SCN-Bz-DTPA:Fibrinogen (10:1, | 1.50 | 1.45 | 103.4 |
| | mol:mol) Collected from PD-10 | | | |
| | column in NS | | | |

### Example 8. Fibrinogen Conjugation with p-SCN-Bz-DTPA and Radiolabeling with ^{99m}Tc

Fibrinogen was conjugated with p-SCN-Bz-DTPA according to the methods described above in Example 7 except that the fibrinogen was prepared for conjugation with p-SCN-Bz-DTPA by first reconstituting 21 mg of the re-lyophilized sample with 2.0 mLs of normal saline under gentle swirling for ˜15-20 min at room temperature. A 1 mL aliquot was loaded onto a PD-10 column (Cytiva G-25 Sephadex; Cat No. 17085101) equilibrated with normal saline and eluted with the same solvent. The protein containing fractions were collected by measuring the UV absorbance at 280 nm. The fibrinogen concentration of pooled fractions was 4.7 mg/mL as determined by UV absorbance at 280 nm and an extinction coefficient of 15.1 for a 10 mg/mL solution of fibrinogen. A calculation was made so that p-SCN-Bz-DTPA (Macrocyclics, Cat no. B-305) was added to fibrinogen in 12:1 and 30:1 molar ratios. The conjugation reaction for a 12:1 p-SCN-Bz-DTPA:Fibrinogen molar ratio was performed by adding 1.06 mLs of fibrinogen solution (14.7 nmol) to a metal-free eppendorf vial followed by addition of 176.5 nmol of p-SCN-Bz-DTPA (23 μl from a 5 mg/mL solution of p-SCN-Bz-DTPA). The pH of the solution was adjusted to 9.1 using sodium carbonate, incubated at 37° C. for 90 minutes, then the pH was lowered to 7.0 with 0.05 N HCl, and stored in a −80° C. freezer for 24 hours. The frozen sample was thawed and an aliquot was analyzed by HPLC to ensure that the protein showed no signs of degradation from the processing steps thus far and as a baseline prior to radiolabeling. The remaining volume was loaded onto a PD-10 column equilibrated with normal saline and eluted with the same solvent for purification of the Fibrinogen conjugate. The protein containing fractions were then collected, pooled, and the protein concentration was determined to be 1.96 mg/mL. The solution was then concentrated to 3.39 mg/mL using a Millipore Amicon Ultra 30 kDa molecular weight cutoff centrifugal filter. A separate conjugation reaction was performed with a 30:1 molar ratio for p-SCN-Bz-DTPA:Fibrinogen by adding 441 nmol of p-SCN-Bz-DTPA (57 μL of a 5 mg/mL p-SCN-Bz-DTPA solution) to 1.06 mL of the same mixture that was used for the 12:1 molar ratio in a metal free eppendorf vial. The pH of the solution was adjusted to 9.1 with sodium carbonate, incubated for 90 minutes at 37° C., lowered to pH 7.0 with 0.05 N HCl, and then stored in a −80° C. freezer for 24 hours. The thawed sample from the 30:1 molar ratio of p-SCN-Bz-DTPA:Fibrinogen was also analyzed by HPLC, loaded onto a PD-10 column, collected, and pooled as described above. The fibrinogen concentration for that sample was 2.16 mg/mL and then concentrated to 3.07 mg/mL using a 30 KDa molecular weight cutoff centrifugal filter unit.

The Fibrinogen conjugates were then radiolabled with ^{99m}Tc. To each reaction vial a total of 1 mCi of ^{99m}Tc was added, followed by a 0.2 mL aliquot of Fibrinogen conjugates produced from the 12:1 and 30:1 p-SCN-Bz-DTPA:Fibrinogen molar ratio experiments to separate vials. One μL of SnCl₂ solution (1 mg/mL) was then added to each reaction vial and the ^{99m}Tc reduction was viewed by TLC using an acetone eluent. The reaction was incubated at room temperature for 20 minutes with occasional gentle mixing and the extent of radiolabeling was assessed using an Agilent HPLC with UV and radiometric detection. Chromatographic separation was performed on a TSK-gel 3000SWXL column from Tosoh Biosciences with column dimensions of 7.8×300 mm, 3-5 μm particle size. The flow rate was 1.0 mL/min with a mobile phase of 100 mM sodium citrate, 100 mM sodium chloride, pH 6.4. Injection volume was 20 μL and the detection wavelength was 280 nm. Also, a sample of Fibrinogen conjugate from the 30:1 p-SCN-Bz-DTPA:Fibrinogen reactions was loaded onto a PD-10 column and eluted with saline in 10 fractions that were measured for radioactivity using a dose calibrator.

HPLC-UV analysis for the conjugates from the 12:1 and 30:1 molar ratio of p-SCN-Bz-DTPA:Fibrinogen at 280 nm showed a major peak at RT ˜6.2-6.3 min, which was fibrinogen main peak. There was a smaller peak that eluted at RT ˜5.7 min, just prior to the fibrinogen main peak. This peak was most likely a high molecular weight form of fibrinogen from dimerization/aggregation during the procedure. Fibrinogen may have aggregated by itself possibly from interaction with p-SCN-Bz-DTPA. Low level impurities were detected at RTs of ˜9 and 11 min. HPLC-radiometric analysis for the 12:1 and 30:1 molar ratio of p-SCN-Bz-DTPA:Fibrinogen showed a peak at RT ˜6.5-6.6 min and 5.9-6.0 minutes that corresponds to the fibrinogen main peak and the fibrinogen aggregate for both conjugates. There were no additional peaks in the chromatogram for HPLC-radiometric detection, suggesting that all of the ^{99m}Tc was bound to fibrinogen and the fibrinogen aggregate. The ^{99m}Tc Fibrinogen conjugate from the 30:1 p-SCN-Bz-DTPA:Fibrinogen molar ratio was loaded onto a PD-10 column equilibrated with normal saline and eluted with the same solvent. The dose calibrator readings of the collected fractions 4-8 (in μCi) further confirmed that the radioactivity elutes with the fibrinogen containing fractions, which are separated from free ^{99m}Tc (Table 16).

TABLE 16
Dose calibrator readings of the collected fractions for
the purification of the ^{99m}Tc Fibrinogen conjugate.

| Fraction No. | 1 | 2 | 3 | 4 | 5 | 6 | 7 | 8 | 9 | 10 |
|---|---|---|---|---|---|---|---|---|---|---|
| ^{99m}Tc (μCi) | 0 | 0 | 0 | 49 | 183 | 62 | 7 | 0.9 | 0.5 | 0 |

### Example 9. SPECT/CT Imaging Studies with ^{99m}Tc-DTPA-DA-Fibrinogen

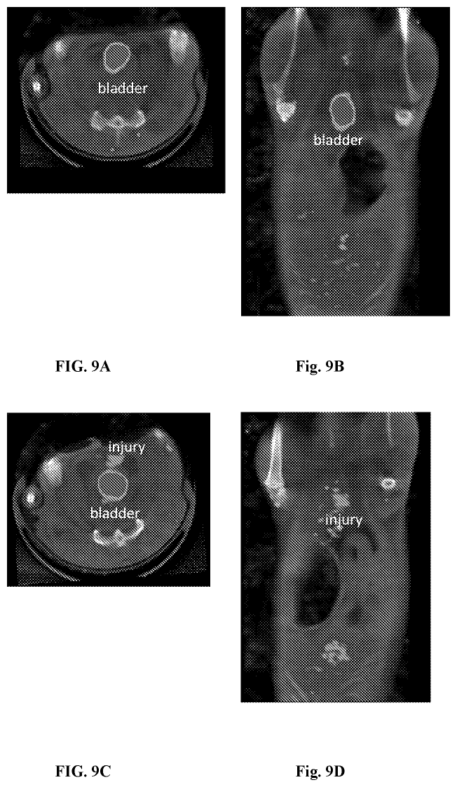

To assess the in vivo efficacy of ^{99m}Tc-DTPA-DA fibrinogen in locating sites of GI bleeding we used colonic injury and sham surgery models. In all models, an incision was made in the abdominal cavity of anesthetized rats. The colonic injury was made by transecting the colon. The sham surgeries were conducted by making the same incision down the midline of the abdominal cavity. After all bleeding in the stomach or colon stopped, the abdominal cavity was closed. Rats were then injected with ˜250 μCi ^{99m}Tc-DTPA-DA Fibrinogen, prepared according to Example 1, and subjected to SPECT/CT imaging 5 mins post infusion. The proposed metrics for success of the tracer were the ability of quantitative data from a SPECT/CT image to identify the injury site. FIGS. 9A-D display representative images from the colon injury model (9C, 9D) versus a sham surgery (9A, 9B). As can be seen in the images, significant ^{99m}Tc-DTPA-DA Fibrinogen uptake is observed at the site of the injury. Negligible uptake is observed in the stomach or colon in the sham surgery animals. The quantified data from both injury models (n=7-9, One way ANOVA *p<0.05 as compared to sham) demonstrated that no appreciable activity was measured in the sham surgery animals. Additionally, significant uptake is observed in both the stomach (2.35 fold over sham) and colon (4.15 fold over sham) injuries. The quantitative data demonstrate that ^{99m}Tc-Fibrinogen specifically binds to the site of injury.

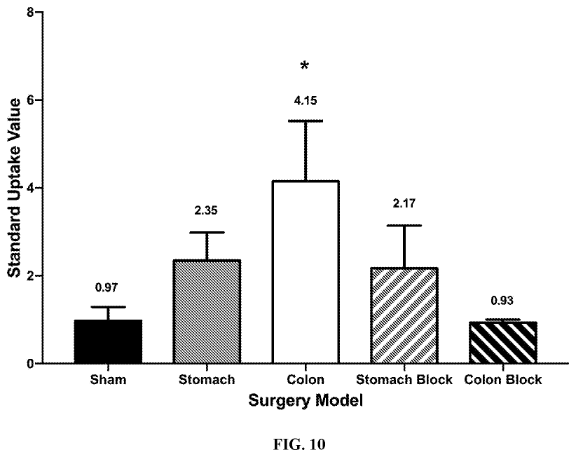

Blocking specificity studies: We performed a blocking study using non-radiolabeled, “cold” fibrinogen administered immediately prior to administration of ^{99m}Tc-DTPA-fibrinogen. Following implementation of the injury model described above, rats were administered 3 mg/kg of reconstituted Fibryga® (Human Fibrinogen, Lyophilized Powder for Reconstitution for Intravenous Use) immediately prior to administration of ˜250 μCi ^{99m}Tc-DTPA-DA fibrinogen, prepared according to Example 1. SPECT/CT analysis was then carried out and standard uptake values calculated from analyzed images. FIG. 10 demonstrates that blocking resulted in reduction of ^{99m}Tc-DTPA-DA-fibrinogen uptake similar to the same level observed in Sham animals (p=0.08 as compared to sham, n=4 for blocking group). These results support the specificity of the ^{99m}Tc-DTPA-DA fibrinogen tracer for identifying sites of bleeding via incorporation into a fibrin clot.

In conclusion, we have performed experiments showing the efficacy of a ^{99m}Tc-DTPA-DA fibrinogen radioligand to locate the site of lower GI bleeding in rats using SPECT/CT imaging. We were able to confirm the in vivo imaging results by ex vivo radiometric analysis. A higher signal was observed for the injury site compared to a control healthy site for all rats with injuries, which was not observed in rats with sham injuries. While a high signal was observed in the kidneys and bladder of all the animals, this signal did not interfere with identifying the injury sites. Specificity of the tracer was verified via blocking studies.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. El-Tawil, A. M., Trends on gastrointestinal bleeding and mortality: where are we standing?World J Gastroenterol, 2012. 18(11): p. 1154-8.
2. Kim, B. S., et al., Diagnosis of gastrointestinal bleeding: A practical guide for clinicians. World J Gastrointest Pathophysiol, 2014. 5(4): p. 467-78.
3. Wilcox, C. M., et al., Mortality associated with gastrointestinal bleeding events: Comparing short-term clinical outcomes of patients hospitalized for upper GI bleeding and acute myocardial infarction in a US managed care setting. Clin Exp Gastroenterol, 2009. 2: p. 21-30.
4. Zuccaro, G., Jr., Management of the adult patient with acute lower gastrointestinal bleeding. American College of Gastroenterology. Practice Parameters Committee. Am J Gastroenterol, 1998. 93(8): p. 1202-8.
5. Adang, R. P., et al., Appropriateness of indications for diagnostic upper gastrointestinal endoscopy: association with relevant endoscopic disease. Gastrointest Endosc, 1995. 42(5): p. 390-7.
6. Strate, L. L. and I. M. Gralnek, ACG Clinical Guideline: Management ofPatients With Acute Lower Gastrointestinal Bleeding. Am J Gastroenterol, 2016. 111(5): p. 755.
7. Strate, L. L. and I. M. Gralnek, ACG Clinical Guideline: Management ofPatients With Acute Lower Gastrointestinal Bleeding. Am J Gastroenterol, 2016. 111(5): p. 755.
8. Jubber, I., et al., Non-visible haematuriafor the Detection ofBladder, Upper Tract, and Kidney Cancer: An Updated Systematic Review andMeta-analysis. Eur Urol, 2019.
9. Willis, G. C. and S. Z. Tewelde, The Approach to the Patient with Hematuria. Emerg Med Clin North Am, 2019. 37(4): p. 755-769.
10. Moreno, J. A., et al., Glomerular Hematuria: Cause or Consequence of Renal Inflammation?Int J Mol Sci, 2019. 20(9).
11. Peterson, L. M. and H. S. Reed, Hematuria. Prim Care, 2019. 46(2): p. 265-273.
12. Lungu et al., Evaluation of Fibrinogen-DTPA-99mTC Biodistribution and Imaging Studies, https:/inis.iaea.org/collection/NCLCollectionStore/_Public/29/061/29061123.pdf?r=1&r=1
13. Riccabona, G., and H. Fill, Beta 2-Microglobulin in vitro and 99mTc-fibrinogen in vivo in the follow-up of renal transplants, Contrib Nephrol, 1984; 42:130-5.
14. U.S. Pat. No. 6,314,314 to Karp, for Method for locating an internal bleeding site in a human body.
15. U.S. Pat. No. 6,056,940 to McBride, et al., for Radiolabeled compounds for thrombus imaging.
16. U.S. Pat. No. 5,968,476 to Dean, et al., for Technetium-99m labeled peptides for thrombus imaging.
17. U.S. Pat. No. 5,208,007 to Mills, for Isotopic tracer composition and method for making and using same.
18. U.S. Pat. No. 4,421,735 to Haber, et al, for Radiolabeled diagnostic compositions and method for making the same.
19. U.S. Pat. No. 4,418,052 to Wong, for Novel diagnostic composition and method for radiologic imaging of fibrinogen deposition in the body.
20. U.S. Pat. No. 4,057,617 to Abamovici, et al., for Method of Labeling proteins with technetium.
21. U.S. Pat. Nos. 9,371,355 and 9,938,318 to Schulz et al., for Process for production of fibrinogen.
22. U.S. Pat. Nos. 10,112,972 and 11,401,300 to Schulz et al., for Process for production of fibrinogen and fibrinogen produced thereby.
23. Neisser-Svae, A. et al., “Differences in the biochemical composition of three plasma derived human fibrinogen concentrates,” Thrombosis Research, 205:44-46 (2021).
24. Schulz, P. M. et al., “Biochemical characterization, stability, and pathogen safety of a new fibrinogen concentrate (fibryga®), Biologicals, 52:72-77 (2018).

It will be understood that various details of the presently disclosed subject matter can be changed without departing from the scope of the subject matter disclosed herein. Furthermore, the foregoing description is for the purpose of illustration only, and not for the purpose of limitation.

1. A kit for preparing a technetium-99m (^{99m}Tc)-labeled composition for targeting an injured site in a blood vessel, comprising:
a human fibrinogen;
a chelating agent selected from the group consisting of diethylene triamine pentaacetate (DTPA) dianhydride, p-SCN-Bz-DTPA, HYNIC, and a combination thereof; and
a reducing agent.

2. A kit for preparing a technetium-99m (^{99m}Tc)-labeled composition for targeting an injured site in a blood vessel, comprising:
a conjugated human fibrinogen, wherein the human fibrinogen is bonded to a chelating agent selected from the group consisting of diethylene triamine pentaacetate (DTPA) dianhydride, p-SCN-Bz-DTPA, HYNIC, and a combination thereof, and
a reducing agent.

3. The kit of claim 1, wherein the reducing agent is stannous chloride.

4. The kit of claim 3, further including a first vial containing the conjugated human fibrinogen or the chelating agent and human fibrinogen; and a second vial containing the stannous chloride.

5. The kit of claim 1, wherein the chelating agent is p-SCN-Bz-DTPA.

6. The kit of claim 1, wherein the molar ratio of the chelating agent to the human fibrinogen is from 1:1 to 30:1.

7. The kit of claim 1, and further comprising one or more components selected from the group consisting of a buffering compound, a compound for adjusting pH, a tonicity agent, and an antioxidant.

8. The kit of claim 1, and further comprising a purification column and optionally including an elution solution.

9. The kit of claim 1, wherein the human fibrinogen, the chelating agent, the reducing agent, or a combination thereof is provided as a lyophilized powder.

10. The kit of claim 1, wherein the human fibrinogen contains from 0.01 to 40% (w/w), from 0.01 to 30% (w/w), from 0.01 to 20% (w/w), from 0.01 to 10% (w/w), from 0.01 to 5.0% (w/w), or from 0.50 to 3.0% (w/w) albumin.

11. The kit of claim 1, wherein the fibrinogen is provided in an amount of about 5-15 mg, the chelating agent is provided in an amount of about 0.1-5 g, and the stannous chloride is provided at a concentration of about 0.001-1.90 mg or 0.005-10 mM.

12. A method for preparing a technetium-99m (^{99m}Tc)-labeled fibrinogen composition for targeting an injured site in a blood vessel, comprising:
preparing or providing a conjugated human fibrinogen and a reducing agent using the kit according to claim 1;
combining the conjugated human fibrinogen with a ^{99m}Tc-containing solution, wherein the reducing agent is combined with the conjugated human fibrinogen before, after, or at the same time as the ^{99m}Tc-containing solution;
incubating the combined conjugated human fibrinogen, reducing agent, and ^{99m}Tc-containing solution to give the ^{99m}Tc-labeled fibrinogen composition; and
optionally checking the radiochemical purity of the ^{99m}Tc-labeled fibrinogen composition.

13. The method of claim 12, wherein the conjugated human fibrinogen is prepared as a solution and the reducing agent is added to the solution before combination with the ^{99m}Tc-containing solution.

14. The method of claim 12, wherein the ^{99m}Tc-containing solution is a Na ^{99m}TcO₄ solution.

15. The method of claim 12, wherein the reducing agent is stannous chloride.

16. The method of claim 12, wherein the human fibrinogen is conjugated to p-SCN-Bz-DTPA.

17. A composition prepared according to the method of claim 12.

18. A method for detecting an internal bleeding site in a subject, comprising:
administering to the subject the composition of claim 17; and
imaging a region of interest of the subject, thereby detecting the internal bleeding site in the subject.

19. The method of claim 18, wherein the imaging is conducted using single-photon emission computed tomography (SPECT).

20. The method of claim 18, wherein the region of interest is within the gastrointestinal (GI) system of the subject.

21. The method according to claim 18, wherein the internal bleeding site has inactive bleeding.
